(19) United States
(12) Patent Application Publication
(10) Pub. No.: US 2007/0060748 A1
(43) Pub. Date: Mar. 15, 2007

(54) Compounds with mixed pde-inhibitory and beta-adrenergic antagonist or partial agonist activity for treatment of heart failure

(76) Inventors: Gregory S. Hamilton, Catonsville, MD (US); H. Jeff Leighton, Boston, MA (US)

Correspondence Address: FOLEY AND LARDNER LLP;SUITE 500, 3000 K STREET NW, WASHINGTON, DC, 20007, US

(21) Appl. No.: 10/547,929

(22) PCT Filed: Nov. 28, 2003
(86) PCT No.: PCT/US03/37812

§ 371 Date: Nov. 22, 2006

Related U.S. Application Data
(60) Provisional application No. 60/429,344, filed on Nov. 27, 2002.

(51) Int. Cl. C07D 487/04; C07D 267/22; C07D 403/02

(52) U.S. Cl. 540/468; 544/238; 544/250; 544/236; 544/281; 544/279; 546/157

(57) ABSTRACT

This invention provides compounds that possess inhibitory activity against β-adrenergic receptors and phosphodiesterase PDE, including phosphodiesterase 3 (PDE3). This invention further provides pharmaceutical compositions comprising such compounds; methods of using such compounds for treating cardiovascular disease, stroke, epilepsy, ophthalmic disorder or migraine; and methods of preparing pharmaceutical compositions and compounds that possess inhibitory activity against β-adrenergic receptors and PDE.

This application claims the benefit of U.S. Provisional Patent Application No. 60/429,344, filed Nov. 27, 2002, the entire contents of which are herein incorporated by reference.

Congestive heart failure affects an estimated 4.8 million Americans with over 400,000 new cases diagnosed each year. Despite incremental advances in drug therapy, the prognosis for patients with advanced heart failure remains poor with annual mortality exceeding 40 percent. Although heart transplantation is an effective therapy for patients with advanced heart failure, less than 2,200 heart transplants are performed annually due to a limited supply of donor organs. Recent analyses indicate that further increases in the incidence and prevalence of advanced heart failure are likely, highlighting the pressing need for novel and effective therapeutic strategies.

During heart failure, there is an alteration of calcium homeostasis, including impaired sarcoplasmic reticulum calcium re-uptake, increased basal (diastolic) calcium levels, decreased peak (systolic) calcium and reduced rate of calcium transients, resulting in a decreased force of contraction and a slowing of relaxation. The end results of these abnormalities in calcium homeostasis are depressed contractile function (decreased contractility and cardiac output), impaired ventricular relaxation, and myocyte loss via ischemia and/or apoptosis-related mechanisms. Disregulation of calcium homeostasis has also been implicated in a number of other disease states, including stroke, epilepsy, ophthalmic disorders, and migraine.

Beta-adrenergic blocking agents are common therapy for patients with mild to moderate chronic heart failure (CHF). Some patients on β-blockers may subsequently decompensate, however, and would need acute treatment with a positive inotropic agent. Phosphodiesterase inhibitors (PDEI), such as milrinone or enoximone, retain their full hemodynamic effects in the face of beta-blockade, because the site of PDEI action (cAMP) is downstream of the β-adrenergic receptor, and because β-antagonism reverses receptor pathway desensitization changes, which are detrimental to phosphodiesterase inhibitor response.

### SUMMARY OF THE INVENTION

This invention provides compounds that possess inhibitory activity against β-adrenergic receptors and phosphodiesterase PDE, including phosphodiesterase 3 (PDE3). This invention further provides pharmaceutical compositions comprising such compounds; methods of using such compounds for treating cardiovascular disease, stroke, epilepsy, ophthalmic disorder or migraine; and methods of preparing pharmaceutical compositions and compounds that possess inhibitory activity against β-adrenergic receptors and PDE.

### DETAILED DESCRIPTION

### DEFINITIONS

“Alkyl radicals” refer to radicals of branched and unbrached saturated hydrocarbon chains comprising a designated number of carbon atoms. For example, C₁-C₉ alkyl radicals designates radicals of straight and branched hydrocarbon chains containing from 1 to 9 carbon atoms and includes all isomers. In some embodiments of the present invention, the alkyl radials are C₁-C₁₂ radicals, and in other embodiments they are C₁-C₆ radicals. In yet other embodiments, the alkyl radicals are chosen from methyl, ethyl, propyl, iso-propyl, butyl, iso-butyl, tert-butyl, n-pentyl, and n-hexyl.

“Alkenyl radicals” refer to radicals of branched and unbranched unsaturated hydrocarbon chains comprising a designated number of carbon atoms. For example, C₂-C₉ alkenyl radicals designates radicals of straight and branched hydrocarbon chains containing from 2 to 9 carbon atoms having at least one double bond and includes all isomers. In some embodiments of the present invention, the alkenyl radicals are C₂-C₆, and in others they are C₃-C₉. In yet other embodiments, the alkenyl radicals are chosen from ethenyl, propenyl, iso-propenyl, butenyl, iso-butenyl, tert-butenyl, n-pentenyl, and n-hexenyl.

“Alkynyl radicals” refer to radicals of branched and unbranched unsaturated hydrocarbon chains comprising a designated number of carbon atoms containing a triple bond between at least two carbon atoms and includes all isomers. For example, a C₂-C₉ alkynyl designates straight and branched hydrocarbon chains containing from 2 to 9 carbon atoms having at least one triple bond and includes all isomers. In some embodiments of the present invention, the alkynyl radicals are C₂-C₆, and in others they are C₃-C₉. In some embodiments, the alkynyl radicals are chosen from ethynyl, propynyl, iso-propynyl, butynyl, iso-butynyl, tert-butynyl, and pentynyl, and hexynyl.

“Alkylene radicals” refer to bivalent radicals of alkanes and includes all isomers.

“Alkenylene radicals” refer to bivalent radicals of alkenes having at least one double bond and includes all isomers.

“Alkynylene radicals” refer to bivalent radicals of alkynes having a triple bond between at least two carbon atoms and includes all isomers.

“Cycloalkyl radicals” refer to cyclic alkyl radicals having a designated number of carbon atoms. For example, C₁-C₈ cycloalkyl radicals designates radicals of straight and branched hydrocarbon chains containing from 1 to 8 carbon atoms and includes all isomers. In some embodiments of the present invention, the cycloalkyl radials are C₁-C₆ radicals, and in other embodiments they are C₁-C₄ radicals. In yet other embodiments, the alkyl radicals are chosen from methylcyclopropane, ethylcyclopropane, propylcyclopropane, butylcyclopropane, pentylcyclopropane, methylcyclobutane, ethylcyclobutane, propylcyclobutane, butylcyclobutane, methylcyclopentane, ethylcyclopentane, propylcyclopentane, methylcyclohexane, ethylcyclohexane, cyclopentyl, cyclobutyl, cycopentyl, cyclohexyl, cycloheptyl, and cyclooctyl.

“Cycloalkenyl radicals” refer to cyclic alkyl radicals having a designated number of carbon atoms and at least one double bond. For example, C₂-C₈ cycloalkenyl radicals designates radicals of straight and branched hydrocarbon chains containing from 2 to 8 carbon atoms, having at least one double bond and includes all isomers. In some embodiments of the present invention, the cycloalkenyl radials are C₂-C₆ radicals. In yet other embodiments, the alkyl radicals are chosen from methylcyclopentene, ethylcyclopentene, propylcyclopentene, methylcyclohexene, ethylcyclohexene, cycopentenyl, cyclohexenyl, cycloheptenyl, and cyclooctenyl.

“Cycloalkynyl radicals” refer to cyclic alkyl radicals having a designated number of carbon atoms and at least one triple bond. For example, C₂-C₈ cycloalkynyl radicals designates radicals of straight and branched hydrocarbon chains containing from 2 to 8 carbon atoms, having at least one triple bond and includes all isomers. In some embodiments of the present invention, the cycloalkynyl radials are C₂-C₆ radicals. In yet other embodiments, the alkyl radicals are chosen from methylcyclohexyne, ethylcyclohexyne, cyclohexynyl, cycloheptenynyl, and cyclooctenynyl.

“Alkylthio” refers to a sulfur substituted alkyl radical.

“Alkoxy” refers to the group —OR, wherein R is an alkyl radical as defined above. In some embodiments of the present invention, R is chosen from branched and unbranched saturated hydrocarbon chains containing from 1 to 9 carbon atoms. In some embodiments, R is chosen from alkyl radicals like C₁-C₆ and C₃-C₉. In yet other embodiments, the alkyl radicals are chosen from methyl, ethyl, propyl, iso-propyl, butyl, iso-butyl, tert-butyl, n-pentyl, and n-hexyl.

“Aryl” refers to aromatic, hydrocarbon cyclic moieties having one or more closed rings. For example, aryl may be chosen from C₆ to C₂₄ and from C₁₀ to C₁₈ aromatic hydrocarbon cyclic moieties. In some embodiments, aryl is chosen from phenyls, benzyls, naphthyls, anthracenyls, phenanthracenyls, and biphenyls. In yet other embodiments, aryl is chosen from phenyl, benzyl, naphthyl, anthracenyl, phenanthracenyl, and biphenyl.

“Heteroaryl” refers to aromatic, cyclic moieties having one or more closed rings with one or more heteroatoms (for example, sulfur, nitrogen or oxygen) in at least one of the rings. For example, heteroaryl may be chosen from 5- to 7-membered monocyclic and bicyclic or 7- to 14-membered bicyclic ring systems containing carbon atoms and 1, 2, 3 or 4 heteroatoms independently chosen from a nitrogen atom, an oxygen atom, and a sulfur atom. In some embodiments, heteroaryl radicals are chosen from pyrroles, furanyls, thiophenes, pyridines and isoxazoles. In yet other embodiments, heteroaryl is chosen from radicals of furans, benzofurans, benzothiophenes, oxazoles, thiazoles, and benzopyrans.

“Halo radicals” refers to fluoro, chloro, bromo, and iodo radicals.

“Substituted phenyl” refers to phenyls that are substituted with one or more substituents. For example, the substituents may be chosen from C₁-C₆ alkyl radicals, C₂-C₆ alkenyl radicals, C₂-C₆ alkynyl radicals, C₁-C₆ alkoxy radicals, C₂-C₆ alkenyloxy radicals, phenoxy, benzyloxy, hydroxy, carboxy, hydroperoxy, carbamido, carbamoyl, carbamyl, carbonyl, carbozoyl, amino, hydroxyamino, formamido, formyl, guanyl, cyano, cyanoamino, isocyano, isocyanato, diazo, azido, hydrazino, triazano, nitrilo, nitro, nitroso, isonitroso, nitrosamino, imino, nitrosimino, oxo, C₁-C₆ alkylthio, sulfamino, sulfamoyl, sulfeno, sulfhydryl, sulfinyl, sulfo, sulfonyl, thiocarboxy, thiocyano, isothiocyano, thioformamido, halo, haloalkyl, chlorosyl, chloryl, perchloryl, trifluoromethyl, iodosyl, iodyl, phosphino, phosphinyl, phospho, phosphono, arsino, selanyl, disilanyl, siloxy, silyl, silylene and carbocyclic and heterocyclic moieties.

“Effective amount” refers to the amount sufficient to produce a desired effect. For example, an effective amount for treating heart failure is an amount sufficient to treat heart failure; an effective amount for treating chronic heart failure is an amount sufficient to treat chronic heart failure; an effective amount for inhibiting PDE is an amount sufficient to inhibit PDE; an effective amount for inhibiting PDE 3 is an amount sufficient to inhibit PDE 3; and an effective amount for inhibiting β-adrenergic receptors is an amount sufficient to inhibit the β-adrenergic receptors.

“Metabolite” refers to a substance produced by metabolism or by a metabolic process.

“Pharmaceutically-acceptable carrier” refers to a pharmaceutically-acceptable materials, compositions, and vehicles, such as liquid and solid fillers, diluents, excipients, and solvent encapsulating materials, involved in carrying or transporting the subject compound from one organ, or portion of the body, to another organ, or portion of the body. Each carrier is “acceptable” in the sense of being compatible with the other ingredients of the formulation and being suitable for use with the patient. A pharmaceutically-acceptable carrier may be active or inactive with respect to the patient. In some embodiements, pharmaceutically-acceptable carrier are chosen from: (1) sugars, such as lactose, glucose and sucrose; (2) starches, such as corn starch and potato starch; (3) cellulose band its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

“Pharmaceutically acceptable equivalent” includes, without limitation, pharmaceutically acceptable salts, hydrates, solvates, metabolites, prodrugs, and isosteres. Many pharmaceutically acceptable equivalents are expected to have the same or similar in vitro or in vivo activity as the compounds of the invention.

“Pharmaceutically acceptable salt” refers to acid and base salts of the inventive compounds, which salts are neither biologically nor otherwise undesirable. In some embodiments, the salts can be formed with acids, and in some embodiments the salts can be formed form acetate, adipate, alginate, aspartate, benzoate, benzenesulfonate, bisulfate butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride hydrobromide, hydroiodide, 2-hydroxyethane-sulfonate, lactate, maleate, methanesulfonate, 2-naphthalenesulfonate, nicotinate, oxalate, thiocyanate, tosylate and undecanoate. In some embodiments, the salts can be formed from base salts, and in other embodiments the salts can be formed from ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine. In some embodiments, the basic nitrogen-containing groups can be quarternized with agents including lower alkyl halides such as methyl, ethyl, propyl and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; and aralkyl halides such as benzyl and phenethyl bromides.

“Prodrug” refers to a derivative of the inventive compounds that undergoes biotransformation, such as metabolism, before exhibiting its pharmacological effect(s). The prodrug is formulated with the objective(s) of improved chemical stability, improved patient acceptance and compliance, improved bioavailability, prolonged duration of action, improved organ selectivity, improved formulation (e.g., increased hydrosolubility), and/or decreased side effects (e.g., toxicity). The prodrug can be readily prepared from the inventive compounds, using conventional methodology described, for instance, in BURGER'S MEDICINAL CHEMISTRY AND DRUG CHEMISTRY (5th ed.), volume 1 at pages 172-178, 949-982 (1995) (the disclosure of which is incorporated herein by reference).

“Isosteres” refer to elements, functional groups, substitutents, molecules or ions having different molecular formulae but exhibiting similar or identical physical properties. For example, tetrazole is an isostere of carboxylic acid because it mimics the properties of carboxylic acid even though they have different molecular formulae. Typically, two isosteric molecules have similar or identical volumes and shapes. Ideally, isosteric compounds should be isomorphic and able to co-crystallize. Other physical properties that isosteric compounds often share include boiling point, density, viscosity and thermal conductivity. However, certain properties may be different, such as dipolar moments, polarity, polarization, size and shape, since the external orbitals may be hybridized differently. The term “isosteres” encompasses “bioisosteres,” which, in addition to their physical similarities, share some biological properties. Typically, bioisosteres interact with the same recognition site or produce broadly similar biological effects.

“Stereoisomers” are isomers that differ only in the arrangement of the atoms in space.

“Enantiomers” are stereoisomers that are non-superimposable mirror images of one another.

“Enantiomer-enriched” is a phrase that denotes a mixture in which one enantiomer predominates.

“Animal” refers to a living organism having sensation and the power of voluntary movement, and which requires for its existence oxygen and organic food. Examples include, without limitation, members of the human, equine, porcine, bovine, murine, canine, and feline species. In the case of a human, an “animal” may also be referred to as a “patient.” “Mammal” refers to a warm-blooded vertebrate animal.

“Treating” refers to: (i) preventing a disease, disorder or condition from occurring in an animal that may be predisposed to the disease, disorder and/or condition but has not yet been diagnosed as having it; (ii) inhibiting a disease, disorder or condition, i.e., arresting its development; and/or (iii) relieving a disease, disorder or condition, i.e., causing regression of the disease, disorder and/or condition.

“Heart failure” refers to the pathophysiologic state in which an abnormality of cardiac function is responsible for the failure of the heart to pump blood at a rate commensurate with the requirements of the metabolizing tissues.

“Congestive heart failure” refers to heart failure that results in the development of congestion and edema in the metabolizing tissues.

“Hypertension” refers to elevation of systemic blood pressure.

“SA/AV node disturbance” refers to an abnormal or irregular conduction and/or rhythm associated with the sinoatrial (SA) node and/or the atrioventricular (AV) node.

“Arrhythmia” refers to abnormal heart rhythm. In arrhythmia, the heartbeats may be too slow, too fast, too irregular or too early. Examples of arrhythmia include, without limitation, bradycardia, fibrillation (atrial or ventricular) and premature contraction.

“Hypertrophic subaortic stenosis” refers to enlargement of the heart muscle due to pressure overload in the left ventricle resulting from partial blockage of the aorta.

“Angina” refers to chest pain associated with partial or complete occlusion of one or more coronary arteries in the heart.

Unless the context clearly dictates otherwise, the definitions of singular terms may be extrapolated to apply to their plural counterparts as they appear in the application; likewise, the definitions of plural terms may be extrapolated to apply to their singular counterparts as they appear in the application.

### Compounds

This invention provides compounds of formula (I)
β-(Ar)ₙ-(L)ₘ-X (I)
or a pharmaceutically acceptable equivalent, an isomer or a mixture of isomers thereof, wherein:

m is chosen from 0 and 1;

n is chosen from 0 and 1;

P is chosen from a 2-amino-1-hydroxyeth-1-yl radical, N-substituted-2-amino-1-hydroxyeth-1-yl radicals, N—N-disubstituted-2-amino-1-hydroxyeth-1-yl radicals, a 3-amino-2-hydroxypropoxy radical, N-substituted-3-amino-2-hydroxypropoxy radicals, and N—N-disubstituted-3-amino-2-hydroxypropoxy radicals;

Ar is chosen from aryl radicals and heteroaryl radicals, which aryl and heteroaryl radicals are optionally substituted with one to three substituent(s) chosen from R₂, R₃, and R₄;

R₂, R₃, and R₄ are independently chosen from C₁-C₈ alkyl radicals, C₂-C₈ alkenyl radicals, C₂-C₈ alkynyl radicals, C₁-C₄ alkylthio groups, C₁-C₄ alkoxy groups, halo radicals, a nitro group, a cyano group, a trifluoromethyl group, —NR₅R₆ groups, acylaminoalkyl radicals, —NHSO₂R₁ groups and —NHCONHR₁ groups, wherein one or more —CH₂— group(s) of the alkyl, alkenyl and alkynyl radicals is/are optionally replaced with —O—, —S—, —SO₂— and/or —NR₅—, and the alkyl, alkenyl and alkynyl radicals are optionally substituted with one or more substituent(s) chosen from an oxo group and a hydroxyl group;

R₅ and R₆ are independently chosen from a lone pair of electrons, a hydrogen radical, C₁-C₈ alkyl radicals, C₂-C₈ alkenyl radicals and C₂-C₈ alkynyl radicals, wherein the alkyl, alkenyl and alkynyl radicals are optionally substituted with a substituent chosen from a phenyl radical and substituted phenyl radicals;

R₁ is chosen from C₁-C₈ alkyl radicals, C₃-C₈ cycloalkyl radicals, C₂-C₈ alkenyl radicals, C₃-C₈ cycloalkenyl radicals, C₂-C₈ alkynyl radicals and C₃-C₈ cycloalkynyl radicals;

L is chosen from a direct bond, C₁-C₁₂ alkylene radicals, C₂-C₁₂ alkenylene radicals and C₂-C₁₂ alkynylene radicals, wherein one or more —CH₂— group(s) of the alkylene, alkenylene and alkynylene radicals is/are optionally replaced with —O—, —S—, —SO₂— and/or —NR₅—, and the alkylene, alkenylene and alkynylene radicals are optionally substituted with one or more substituent(s) independently chosen from an oxo group and a hydroxyl group; and

X is chosen from moieties of formulas A-Q: 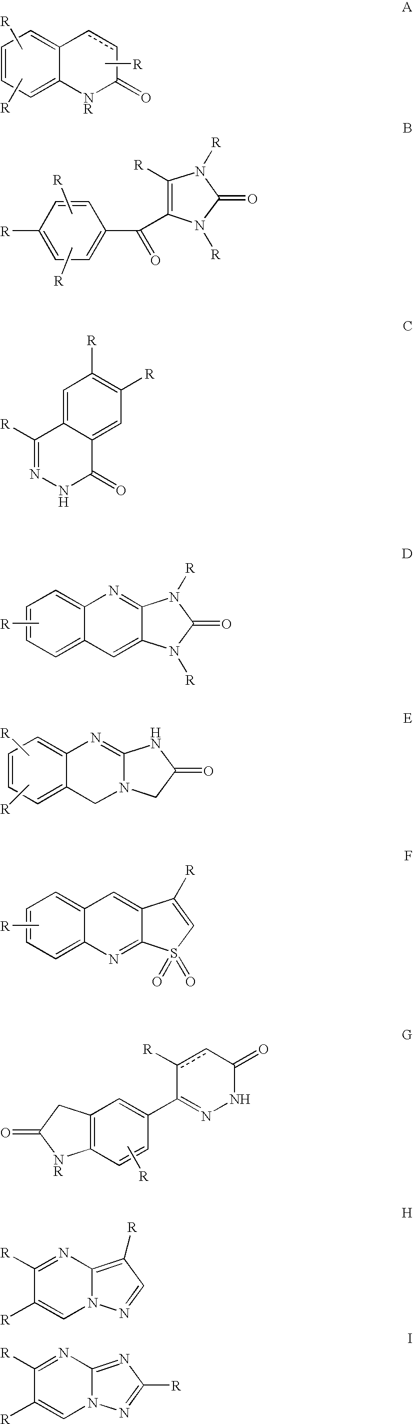 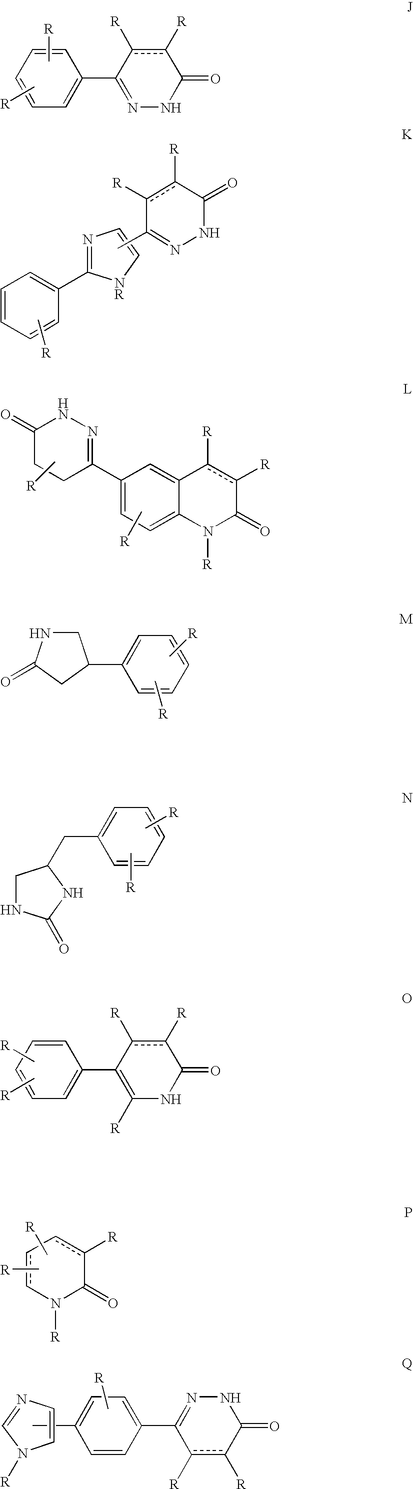
wherein one R group of moieties A-Q forms a covalent bond between X and L when m is 1, or between X and Ar when n is 1 and m is 0, or between X and β when n is 0 and m is 0; and each remaining R group of moieties A-Q is independently chosen from a hydrogen radical, halo radicals, a nitro group, a cyano group, a trifluoromethyl group, an amino group, NR₅R₆ groups, C₁-C₄ alkoxy radicals, C₁-C₄ alkylthio radicals, COOR, radicals, C₁-C₁₂ alkyl radicals, C₂-C₁₂ alkenyl radicals and C₂-C₁₂ alkynyl radicals, wherein one or more —CH₂— group(s) of the alkyl, alkenyl and alkynyl radicals is/are optionally replaced with —O—, —S—, —SO₂— and/or —NR₅—, and the alkyl, alkenyl and alkynyl radicals are optionally substituted with one or more substituent(s) chosen from an oxo group and a hydroxyl group; and
   with the following provisos:
(a) when m+n is 0, when X is chosen from A moieties, when β is chosen from a 2-amino-1-hydroxyeth-1-yl radical, N-substituted-2-amino-1-hydroxyeth-1-yl radicals, and N—N-disubstituted-2-amino-1-hydroxyeth-1-yl radicals, and
   (i) when β is at position 3 or 4 of A, 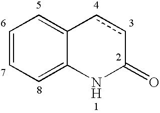
then the N-substituted-2-amino-1-hydroxyeth-1-yl radicals are not substituted with an alkyl radical, a cycloalkyl radical; an alkenyl radical; a cycloalkenyl radical, or an alkynyl radical; and then one substituent of the N—N-disubstituted-2-amino-1-hydroxyeth-1-yl radicals is not an alkyl radical, a cycloalkyl radical; an alkenyl radical; a cycloalkenyl radical, or an alkynyl radical;
   (ii) when β is at position 5 of A, then position 8 of A is not substituted with an alkoxy radical or a hydroxyl radical;
   (iii) when β is at position 6 of A, position 8 of A is not substituted with an alkoxy radical, an acyloxy radical, or a hydroxyl radical; and
   (iv) when β is at position 8 of A and position 5 of A is substituted with an alkoxy radical or a hydroxy radical, then the N-substituted-2-amino-1-hydroxyeth-1-yl radicals are not substituted with an alkyl radical or a cycloalkyl radical;
   and then one substituent of the N—N-disubstituted-2-amino-1-hydroxyeth-1-yl radicals is not an alkyl radical or a cycloalkyl radical
(b) when m+n is 0, when X is chosen from A moieties, when β is chosen from a 3-amino-2-hydroxypropoxy radical, N-substituted-3-amino-2-hydroxypropoxy radicals, and N—N-disubstituted-3-amino-2-hydroxypropoxy radicals, and
   (i) when β is at position 4 of A, then any R attached to the ring nitrogen is not a C₁-C₃ alkyl radical or a C₁-C₃ alkenyl radical;
   (ii) when β is at any position 5-8 of A, then the N-substituted-3-amino-2-hydroxypropoxy radicals are not substituted with an alkyl radical; a cycloalkyl radical; an alkenyl radical; a cycloalkenyl radical; or an alkynyl radical;
   and then one substituent of the N—N-disubstituted-3-amino-2-hydroxypropoxy radicals is not an alkyl radical; a cycloalkyl radical;
   an alkenyl radical; a cycloalkenyl radical; or an alkynyl radical;
(c) when m is 1, when n is 0, when X is chosen from A moieties, when β is chosen from a 3-amino-2-hydroxypropoxy radical, N-substituted-3-amino-2-hydroxypropoxy radicals, and N—N-disubstituted-3-amino-2-hydroxypropoxy radicals, and when β is at position 5 of A, and position 8 of A is substituted with a hydrogen radical, an alkoxy radical, or an aryloxy radical, and the R attached to the ring nitrogen is a hydrogen radical or an alkyl radical, then L is not a C₃ alkenyl radical; and
(d) when m+n is 0, when X is chosen from J moieties, when β is chosen from a 3-amino-2-hydroxypropoxy radical, N-substituted-3-amino-2-hydroxypropoxy radicals, and N—N-disubstituted-3-amino-2-hydroxypropoxy radicals, and when β is attached to the phenyl ring of J, then the N-substituted-3-amino-2-hydroxypropoxy radicals and the N—N-disubstituted-3-amino-2-hydroxypropoxy radicals are not substituted with a C₃-C₄ alkyl radical or a phenethyl radical.

Every variable substituent is defined independently at each occurrence. Thus, the definition of a variable substituent in one part of a formula is independent of its definition(s) elsewhere in that formula and of its definition(s) in other formulas.

In formula (I), moieties A, G, J-L, and O-Q contain dashed lines in their respective structures. These dashed lines indicate that saturation is optional.

In formula (I)'s β, the N-substituted-2-amino-1-hydroxyeth-1-yl radicals, the N—N-disubstituted-2-amino-1-hydroxyeth-1-yl radicals, the N-substituted-3-amino-2-hydroxypropoxy radicals, and N—N-disubstituted-3-amino-2-hydroxypropoxy radicals may be substituted with any group capable of bonding to such radicals.

In some embodiments, formula (I)'s L is chosen from C₁-C₁₂ alkylene radicals, C₂-C₁₂ alkenylene radicals, and C₂-C₁₂ alkynylene radicals. In some embodiments, formula (I)'s L is chosen from C₁-C₈ alkylene radicals, C₂-C₈ alkenylene radicals, and C₂-C₈ alkynylene radicals. In some embodiments, one or more —CH₂— group(s) of the alkylene, alkenylene and alkynylene radicals is/are optionally replaced with —O— and/or —NR₅—, and the alkylene radicals are optionally substituted with one or more oxo group(s). In some embodiments, formula (I)'s L is chosen from C₁-C₈ alkylene radicals. In some embodiments, formula (I)'s L is chosen from —(CH₂)₃O—, —O(CH₂)₃NH(CO)CH₂O—, and —O(CH₂)₃NH(CO)(CH₂)₃O—.

In some embodiments, formula (I)'s X is chosen from moieties of formulas B, E, and O. In some embodiments, formula (I)'s X is chosen from moieties of formula A, when n is 1. In some embodiments, formula (I)'s X is chosen from moieties of formula J, when m+n is 1 or 2.

In some embodiments, formula (I)'s R groups of moieties A-Q are independently chosen from a hydrogen radical; C₁-C₁₂ alkyl radicals; C₂-C₁₂ alkenyl radicals; C₂-C₁₂ alkynyl radicals, halo radicals and cyano group. In some embodiments, formula (I)'s R groups of moieties A-Q are independently chosen from a hydrogen radical; C₁-C₆ alkyl radicals; C₂-C₆ alkenyl radicals; C₂-C₆ alkynyl radicals, halo radicals and cyano group.

In some embodiments, formula (I)'s R₁ is chosen from C₁-C₆ alkyl radicals, C₁-C₆ cycloalkyl radicals, C₂-C₆ alkenyl radicals, C₂-C₆ cycloalkenyl radicals, and C₂-C₆ alkynyl radicals.

In some embodiments, formula (I)'s R₂ is chosen from a cyano group; a nitro group; halo radicals; a hydrogen radical; a trifluoromethyl group; acylaminoalkyl radicals, C₁-C₄ alkoxy groups; C₁-C₄ alkylthio groups; C₁-C₈ alkyl radicals; C₂-C₈ alkenyl radicals; and C₂-C₈ alkynyl radicals. In some embodiments, the acylaminoalkyl radicals contain an alkyl chain having from C₁-C₆.

In some embodiments, formula (I)'s R₃ is chosen from a cyano group; a nitro group; halo radicals; a hydrogen radical; a trifluoromethyl group; acylaminoalkyl radicals, C₁-C₄ alkoxy groups; C₁-C₄ alkylthio groups; C₁-C₈ alkyl radicals; C₂-C₈ alkenyl radicals; and C₂-C₈ alkynyl radicals. In some embodiments, the acylaminoalkyl radicals contain an alkyl chain having from C₁-C₆.

In some embodiments, formula (I)'s R₄ is chosen from a cyano group; a nitro group; halo radicals; a hydrogen radical; a trifluoromethyl group; acylaminoalkyl radicals, C₁-C₄ alkoxy groups; C₁-C4 alkylthio groups; C₁-C₈ alkyl radicals; C₂-C₈ alkenyl radicals; and C₂-C₈ alkynyl radicals. In some embodiments, the acylaminoalkyl radicals contain an alkyl chain having from C₁-C₆.

In some embodiments, formula (I)'s R₅ is chosen from a lone pair of electrons; a hydrogen radical; C₁-C₈ alkyl radicals; C₂-C₈ alkenyl radicals; and C₂-C₈ alkynyl radicals.

In some embodiments, formula (I)'s R₆ is chosen from a lone pair of electrons; a hydrogen radical; C₁-C₈ alkyl radicals; C₂-C₈ alkenyl radicals; and C₂-C₈ alkynyl radicals.

In some embodiments, formula (I)'s Ar is chosen from phenyl radicals, naphthyl radicals, pyridyl radicals, isoxazoyl radicals, pyridyl radicals, quinolyl radicals, and isoquinolyl radicals. In other embodiments, the heteroaryl radicals are chosen from radicals of furans, benzofurans, benzothiophenes, oxazoles, thiazoles, and benzopyrans. In some embodiments, formula (I)'s Ar is chosen from groups Ar₁—Ar₇: 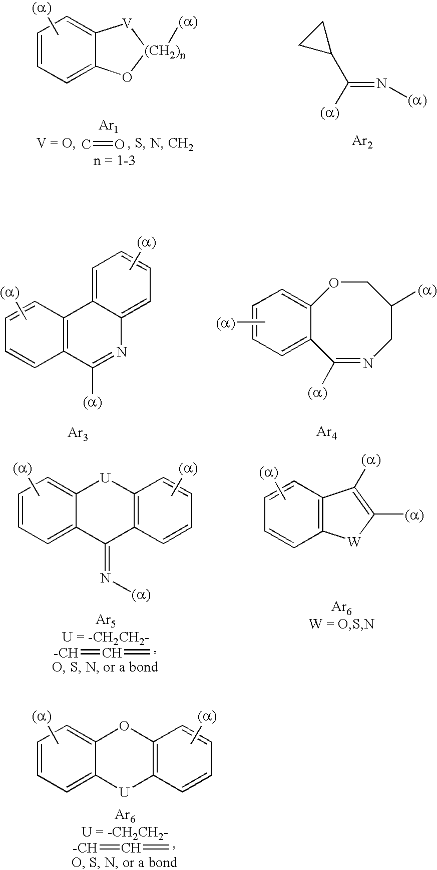
wherein (α) indicates the position where Ar may bond to β, L, and X.

Since the compounds of the present invention may possess one or more asymmetric carbon center(s), they may be capable of existing in the form of optical isomers as well as in the form of racemic or non-racemic mixtures of optical isomers. The optical isomers can be obtained by resolution of the racemic mixtures according to conventional processes. One such process entails formation of diastereoisomeric salts, by treatment with an optically active acid or base, and then separation of the mixture of diastereoisomers by crystallization, followed by liberation of the optically active bases from these salts. Examples of appropriate acids are tartaric, diacetyltartaric, dibenzoyltartaric, ditoluoyltartaric, and camphorsulfonic acid.

A different process for separating optical isomers involves the use of a chiral chromatography column optimally chosen to maximize the separation of the enantiomers. Still another available method involves synthesis of covalent diastereoisomeric molecules, for example, esters, amides, acetals, and ketals, by reacting the compounds of the present invention with an optically active acid in an activated form, an optically active diol or an optically active isocyanate. The synthesized diastereoisomers can be separated by conventional means such as chromatography, distillation, crystallization or sublimation, and then hydrolyzed to deliver the enantiomerically pure compound. In some cases hydrolysis to the “parent” optically active drug is not necessary prior to dosing the patient, since the compound can behave as a prodrug. The optically active compounds of the present invention likewise can be obtained by utilizing optically active starting materials.

It is understood that the compounds of the present invention encompass individual optical isomers as well as racemic and non-racemic mixtures.

Accordingly, in some embodiments, formula (I)'s β is chosen from a 2-amino-1-hydroxyeth-1-yl radical, N-substituted-2-amino-1-hydroxyeth-1-yl radicals, and N—N-disubstituted-2-amino-1-hydroxyeth-1-yl radicals, wherein the carbon at position 1 of each radical is enriched over its mirror image counterpart. In some embodiments, the R configuration is enriched.

In some embodiments, formula (I)'s β is chosen from a 3-amino-2-hydroxypropoxy radical, N-substituted-3-amino-2-hydroxypropoxy radicals, and N—N-disubstituted-3-amino-2-hydroxypropoxy radicals, wherein the carbon at position 2 of each radical is enriched over its mirror image counterpart. In some embodiments, the S configuration is enriched.

In some embodiments, m+n is 0. In other embodiments, m+n is 1. In other embodiments, m+n is 2.

In another embodiment, a compound of present invention is chosen from those of formula (I) as defined above, pharmaceutically acceptable equivalents and stereoisomers thereof, wherein:

m is chosen from 0 and 1;

n is chosen from 0 and 1;

β is chosen from radicals of formula (β₁) and radicals of formula (β₂):
—CHOHCH₂NZ₁Z₂ (β₁) and
OCH₂CHOHCH₂NZ₁Z₂ (β₂);
wherein Z₁ and Z₂ are independently chosen from a hydrogen radical, R₁ radicals, and —CH₂CH₂—Y—R₁ radicals;
   wherein R₁ is as defined above;
   wherein Y is chosen from a —NHCO— radical, a —NHCONH— radical, and a —NHSO₂— radical;
Ar is as defined above;
L is as defined above; and
X is as defined above;
   with the following provisos:
(a) when m+n is 0, when X is chosen from A moieties, when β is chosen from β₁ radicals, and
   (i) when β₁ is at position 3 or 4 of A, 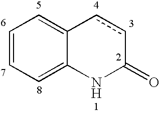
then one of β₁'s Z, or Z₂ is not an R₁ radical;
   (ii) when β₁ is at position 5 of A, then position 8 of A is not substituted with an alkoxy radical or a hydroxyl radical;
   (iii) when β₁ is at position 6 of A, position 8 of A is not substituted with an alkoxy radical, an acyloxy radical, or a hydroxyl radical; and
   (iv) when β₁ is at position 8 of A and position 5 is substituted with an alkoxy radical or a hydroxy radical, then one of β₁'s Z₁ or Z₂ is not an alkyl radical or a cycloalkyl radical;
(b) when m+n is 0, when X is chosen from A moieties, when β is chosen from β₂, and
   (i) when β₂ is at position 4 of A, then any R attached to the ring nitrogen is not a C₁-C₃ alkyl radical or a C₁-C₃ alkenyl radical;
   (ii) when β₂ is at any position 5-8 of A, then one of β₂'s Z₁ or Z₂ is not an alkyl radical; a cycloalkyl radical; an alkenyl radical; a cycloalkenyl radical; or an alkynyl radical;
(c) when m is 1, when n is 0, when X is chosen from moieties of formula A, when L is attached to position 5 of A, when position 8 of A is substituted with a hydrogen radical, an alkoxy radical, or an aryloxy radical, and when the R attached to the ring nitrogen is a hydrogen radical or an alkyl radical, then L is not a C₃ alkenyl radical; and
(d) when m+n is 0, when X is chosen from J moieties, when β is chosen from β₂, when β₂ is attached to the phenyl ring of J, then β₂'s Z₁ and Z₂ are not a C₃-C₄ alkyl radical or a phenethyl radical.

In some embodiments, formula (I)'s L is chosen from C₁-C₁₂ alkylene radicals, C₂-C₁₂ alkenylene radicals, and C₂-C₁₂ alkynylene radicals. In some embodiments, formula (I)'s L is chosen from C₁-C₈ alkylene radicals, C₂-C₈ alkenylene radicals, and C₂-C₈ alkynylene radicals. In some embodiments, one or more —CH₂— group(s) of the alkylene, alkenylene and alkynylene radicals is/are optionally replaced with —O— and/or —NR₅—, and the alkylene radicals are optionally substituted with one or more oxo group(s). In some embodiments, formula (I)'s L is chosen from C₁-C₈ alkylene radicals. In some embodiments, formula (I)'s L is chosen from —O(CH₂)₃O—, —O(CH₂)₃NH(CO)CH₂O—, and —O(CH₂)₃NH(CO)(CH₂)₃O—.

In some embodiments, formula (I)'s X is chosen from moieties of formulas B, E, and O. In some embodiments, formula (I)'s X is chosen from moieties of formula A, when n is 1. In some embodiments, formula (I)'s X is chosen from moieties of formula J, when m+n is 1 or 2.

In some embodiments, formula (I)'s R groups of moieties A-Q are independently chosen from a hydrogen radical; C₁-C₁₂ alkyl radicals; C₂-C₁₂ alkenyl radicals; and C₂-C₁₂ alkynyl radicals.) In some embodiments, formula (I)'s R groups of moieties A-Q are independently chosen from a hydrogen radical; C₁-C₆ alkyl radicals; C₂-C₆ alkenyl radicals; and C₂-C₆ alkynyl radicals.

In some embodiments, formula (I)'s R₁ is chosen from C₁-C₆ alkyl radicals, C₁-C₆ cycloalkyl radicals, C₂-C₆ alkenyl radicals, C₂-C₆ cycloalkenyl radicals, and C₂-C₆ alkynyl radicals.

In some embodiments, formula (I)'s R₂ is chosen from a cyano group; a nitro group; halo radicals; a hydrogen radical; a trifluoromethyl group; acylaminoalkyl radicals, C₁-C₄ alkoxy groups; C₁-C₄ alkylthio groups; C₁-C₈ alkyl radicals; C₂-C₈ alkenyl radicals; and C₂-C₈ alkynyl radicals. In some embodiments, the acylaminoalkyl radicals contain an alkyl chain having from C₁-C₆.

In some embodiments, formula (I)'s R₃ is chosen from a cyano group; a nitro group; halo radicals; a hydrogen radical; a trifluoromethyl group; acylaminoalkyl radicals, C₁-C₄ alkoxy groups; C₁-C₄ alkylthio groups; C₁-C₈ alkyl radicals; C₂-C₈ alkenyl radicals; and C₂-C₈ alkynyl radicals. In some embodiments, the acylaminoalkyl radicals contain an alkyl chain having from C₁-C₆.

In some embodiments, formula (I)'s R₄ is chosen from a cyano group; a nitro group; halo radicals; a hydrogen radical; a trifluoromethyl group; acylaminoalkyl radicals, C₁-C₄ alkoxy groups; C₁-C₄ alkylthio groups; C₁-C₈ alkyl radicals; C₂-C₈ alkenyl radicals; and C₂-C₈ alkynyl radicals. In some embodiments, the acylaminoalkyl radicals contain an alkyl chain having from C₁-C₆.

In some embodiments, formula (I)'s R₅ is chosen from a lone pair of electrons; a hydrogen radical; C₁-C₈ alkyl radicals; C₂-C₈ alkenyl radicals; and C₂-C₈ alkynyl radicals.

In some embodiments, formula (I)'s R₆ is chosen from a lone pair of electrons; a hydrogen radical; C₁-C₈ alkyl radicals; C₂-C₈ alkenyl radicals; and C₂-C₈ alkynyl radicals.

In some embodiments, formula (I)'s Ar is chosen from phenyl radicals, naphthyl radicals, pyridyl radicals, isoxazoyl radicals, pyridyl radicals, quinolyl radicals, and isoquinolyl radicals. In other embodiments, Ar is a heteroaryl chosen from radicals of furans, benzofurans, benzothiophenes, oxazoles, thiazoles, and benzopyrans. In some embodiments, formula (I)'s Ar is chosen from groups Ar₁—Ar₇ as defined above.

In some embodiments, the compound of the present invention is chosen from pharmaceutically acceptable salts of compounds of formula (I).

In some embodiments, the compound of the present invention is chosen from hydrates of compounds of formula (I).

In some embodiments, the compound of the present invention is chosen from solvates of compounds of formula (I).

In some embodiments, the compound of the present invention is chosen from metabolites of compounds of formula (I).

In some embodiments, the compound of the present invention is chosen from prodrugs of compounds of formula (I).

In some embodiments, the compound of the present invention is chosen from isosteres of compounds of formula (I).

In some embodiments, formula (I)'s Z₁ and Z₂ are the same. In other embodiments, in formula (II), Z₁ and Z₂ differ. In some embodiments, formula (I)'s Z₁ and Z₂ are chosen from R₁ radicals, and in other embodiments, formula (I)'s Z₁ and Z₂ are chosen from —CH₂CH₂—Y—R, radicals.

In some embodiments, formula (I)'s β is chosen from radicals of formula (β₁*) and radicals of formula (β₂*):
—C*HOHCH₂NZ₁Z₂ (β₁*) and
—OCH₂C*HOHCH₂NZ₁Z₂ (β₂*);
wherein the * on the Cs in β₁* and β₂* denote chiral centers that are enriched over their respective mirror image counterparts. In some embodiments, formula (I)'s * on the C in β₁* denotes a chiral-carbon center that is enriched in the R configuration. In some embodiments, formula (I)'s * on the C in β₂* denotes a chiral-carbon center that is enriched in the S configuration.

In some embodiments, m+n is 0. In other embodiments, m+n is 1. In other embodiments, m+n is 2.

In another embodiment, a compound of present invention is chosen from those of formula (I) as defined above, pharmaceutically acceptable equivalents and stereoisomers thereof, wherein:

m is chosen from 0 and 1;

n is chosen from 0 and 1;

β is chosen from radicals of formula (β₁) and radicals of formula (β₂) as defined above;

Ar is as defined above;

L is chosen from a —CH₂CH₂— radical, a —CH(CH₃)CH₂— radical, and a —CH(CH₃)₂CH₂— radical; and

X is as defined above.

In some embodiments, formula (I)'s R groups of moieties of formula B—I and K-Q are independently chosen from a hydrogen radical; C₁-C₁₂ alkyl radicals; C₂-C₁₂ alkenyl radicals; and C₂-C₁₂ alkynyl radicals. In some embodiments, formula (I)'s R groups of moieties of formula B—I and K-Q are independently chosen from a hydrogen radical; C₁-C₆ alkyl radicals; C₂-C₆ alkenyl radicals; and C₂-C₆ alkynyl radicals.

In some embodiments, formula (I)'s R₁ is chosen from C₁-C₆ alkyl radicals, C₁-C₆ cycloalkyl radicals, C₂-C₆ alkenyl radicals, C₂-C₆ cycloalkenyl radicals, and C₂-C₆ alkynyl radicals.

In some embodiments, formula (I)'s R₂ is chosen from a cyano group; a nitro group; halo radicals; a hydrogen radical; a trifluoromethyl group; acylaminoalkyl radicals, C₁-C₄ alkoxy groups; C₁-C₄ alkylthio groups; C₁-C₈ alkyl radicals; C₂-C₈ alkenyl radicals; and C₂-C₈ alkynyl radicals. In some embodiments, the acylaminoalkyl radicals contain an alkyl chain having from C₁-C₆.

In some embodiments, formula (I)'s R₃ is chosen from a cyano group; a nitro group; halo radicals; a hydrogen radical; a trifluoromethyl group; acylaminoalkyl radicals, C₁-C₄ alkoxy groups; C₁-C₄ alkylthio groups; C₁-C₈ alkyl radicals; C₂-C₈ alkenyl radicals; and C₂-C₈ alkynyl radicals. In some embodiments, the acylaminoalkyl radicals contain an alkyl chain having from C₁-C₆.

In some embodiments, formula (I)'s R₄ is chosen from a cyano group; a nitro group; halo radicals; a hydrogen radical; a trifluoromethyl group; acylaminoalkyl radicals, C₁-C₄ alkoxy groups; C₁-C₄ alkylthio groups; C₁-C₈ alkyl radicals; C₂-C₈ alkenyl radicals; and C₂-C₈ alkynyl radicals. In some embodiments, the acylaminoalkyl radicals contain an alkyl chain having from C₁-C₆.

In some embodiments, formula (I)'s R₅ is chosen from a lone pair of electrons; a hydrogen radical; C₁-C₈ alkyl radicals; C₂-C₈ alkenyl radicals; and C₂-C₈ alkynyl radicals.

In some embodiments, formula (I)'s R₆ is chosen from a lone pair of electrons; a hydrogen radical; C₁-C₈ alkyl radicals; C₂-C₈ alkenyl radicals; and C₂-C₈ alkynyl radicals.

In some embodiments, formula (I)'s Ar is chosen from phenyl radicals, naphthyl radicals, pyridyl radicals, isoxazoyl radicals, pyridyl radicals, quinolyl radicals, and isoquinolyl radicals. In other embodiments, Ar is a heteroaryl chosen from radicals of furans, benzofurans, benzothiophenes, oxazoles, thiazoles, and benzopyrans. In some embodiments, formula (I)'s Ar is chosen from groups Ar₁—Ar₇ as defined above.

In some embodiments, the compound of the present invention is chosen from pharmaceutically acceptable salts of compounds of formula (I).

In some embodiments, the compound of the present invention is chosen from hydrates of compounds of formula (I).

In some embodiments, the compound of the present invention is chosen from solvates of compounds of formula (I).

In some embodiments, the compound of the present invention is chosen from metabolites of compounds of formula (I).

In some embodiments, the compound of the present invention is chosen from prodrugs of compounds of formula (I).

In some embodiments, the compound of the present invention is chosen from isosteres of compounds of formula (I).

In some embodiments, formula (I)'s Z₁ and Z₂ are the same. In other embodiments, in formula (II), Z₁ and Z₂ differ. In some embodiments, formula (I)'s Z₁ and Z₂ are chosen from R₁ radicals, and in other embodiments, formula (I)'s Z₁ and Z₂ are chosen from —CH₂CH₂—Y—R₁ radicals.

In some embodiments, formula (I)'s β is chosen from radicals of formula (β₁*) and radicals of formula (β₂*) as defined above. In some embodiments, formula (I)'s * on the C in β₁* denotes a chiral-carbon center that is enriched in the R configuration. In some embodiments, formula (I)'s * on the C in β₂* denotes a chiral-carbon center that is enriched in the S configuration.

In some embodiments, m+n is 0. In other embodiments, m+n is 1. In other embodiments, m+n is 2.

In another embodiment, a compound of present invention is chosen from those of formula (I) as defined above, pharmaceutically acceptable equivalents and stereoisomers thereof, wherein:

β is chosen from radicals of formula (PI) and radicals of formula (02) as defined above;

Ar is as defined above;

L is chosen from a —CH₂CH₂— radical, a —CH(CH₃)CH₂— radical, and a —CH(CH₃)₂CH₂— radical; and

X is as defined above.

In some embodiments, formula (I)'s R groups of moieties of formula B, E and O are independently chosen from a hydrogen radical; C₁-C₁₂ alkyl radicals; C₂-C₁₂ alkenyl radicals; and C₂-C₁₂ alkynyl radicals. In some embodiments, formula (I)'s R groups of moieties of formula B, E and O are independently chosen from a hydrogen radical; C₁-C₆ alkyl radicals; C₂-C₆ alkenyl radicals; and C₂-C₆ alkynyl radicals.

In some embodiments, formula (I)'s R₁ is chosen from C₁-C₆ alkyl radicals, C₁-C₆ cycloalkyl radicals, C₂-C₆ alkenyl radicals, C₂-C₆ cycloalkenyl radicals, and C₂-C₆ alkynyl radicals.

In some embodiments, formula. (I)'s R₂ is chosen from a cyano group; a nitro group; halo radicals; a hydrogen radical; a trifluoromethyl group; acylaminoalkyl radicals, C₁-C₄ alkoxy groups; C₁-C₄ alkylthio groups; C₁-C₈ alkyl radicals; C₂-C₈ alkenyl radicals; and C₂-C₈ alkynyl radicals. In some embodiments, the acylaminoalkyl radicals contain an alkyl chain having from C₁-C₆.

In some embodiments, formula (I)'s R₃ is chosen from a cyano group; a nitro group; halo radicals; a hydrogen radical; a trifluoromethyl group; acylaminoalkyl radicals, C₁-C₄ alkoxy groups; C₁-C₄ alkylthio groups; C₁-C₈ alkyl radicals; C₂-C₈ alkenyl radicals; and C₂-C₈ alkynyl radicals. In some embodiments, the acylaminoalkyl radicals contain an alkyl chain having from C₁-C₆.

In some embodiments, formula (I)'s R₄ is chosen from a cyano group; a nitro group; halo radicals; a hydrogen radical; a trifluoromethyl group; acylaminoalkyl radicals, C₁-C₄ alkoxy groups; C₁-C₄ alkylthio groups; C₁-C₈ alkyl radicals; C₂-C₈ alkenyl radicals; and C₂-C₈ alkynyl radicals. In some embodiments, the acylaminoalkyl radicals contain an alkyl chain having from C₁-C₆.

In some embodiments, formula (I)'s R₅ is chosen from a lone pair of electrons; a hydrogen radical; C₁-C₈ alkyl radicals; C₂-C₈ alkenyl radicals; and C₂-C₈ alkynyl radicals.

In some embodiments, formula (I)'s R₆ is chosen from a lone pair of electrons; a hydrogen radical; C₁-C₈ alkyl radicals; C₂-C₈ alkenyl radicals; and C₂-C₈ alkynyl radicals.

In some embodiments, formula (I)'s Ar is chosen from phenyl radicals, naphthyl radicals, pyridyl radicals, isoxazoyl radicals, pyridyl radicals, quinolyl radicals, and isoquinolyl radicals. In other embodiments, Ar is a heteroaryl chosen from radicals of furans, benzofurans, benzothiophenes, oxazoles, thiazoles, and benzopyrans. In some embodiments, formula (I)'s Ar is chosen from groups Ar₁—Ar₇ as defined above.

In some embodiments, the compound of the present invention is chosen from pharmaceutically acceptable salts of compounds of formula (I).

In some embodiments, the compound of the present invention is chosen from hydrates of compounds of formula (I).

In some embodiments, the compound of the present invention is chosen from solvates of compounds of formula (I).

In some embodiments, the compound of the present invention is chosen from metabolites of compounds of formula (I).

In some embodiments, the compound of the present invention is chosen from prodrugs of compounds of formula (I).

In some embodiments, the compound of the present invention is chosen from isosteres of compounds of formula (I).

In some embodiments, formula (I)'s Z₁ and Z₂ are the same. In other embodiments, in formula (II), Z₁ and Z₂ differ. In some embodiments, formula (I)'s Z₁ and Z₂ are chosen from R₁ radicals, and in other embodiments, formula (I)'s Z₁ and Z₂ are chosen from —CH₂CH₂—Y—R₁ radicals.

In some embodiments, formula (I)'s β is chosen from radicals of formula (β₁*) and radicals of formula (β₂*) as defined above. In some embodiments, formula (I)'s * on the C in β₁* denotes a chiral-carbon center that is enriched in the R configuration. In some embodiments, formula (I)'s * on the C in β₂* denotes a chiral-carbon center that is enriched in the S configuration.

In some embodiments, m+n is 0. In other embodiments, m+n is 1. In other embodiments, m+n is 2.

In another embodiment of the present invention, a compound of the present invention is chosen from compounds containing a radical β and a radical X, wherein:

β is chosen from a 2-amino-1-hydroxyeth-1-yl radical, N-substituted-2-amino-1-hydroxyeth-1-yl radicals, N—N-disubstituted-2-amino-1-hydroxyeth-1-yl radicals, a 3-amino-2-hydroxypropoxy radical, N-substituted-3-amino-2-hydroxypropoxy radicals, and N—N-disubstituted-3-amino-2-hydroxypropoxy radicals, wherein the N—N-disubstituted-radicals are substituted with identical substituents.

In some embodiments, β is chosen from radicals of formula (β₁) and radicals of formula (β₂) as defined above. In some embodiments, β is chosen from radicals of formula (β₁*) and radicals of formula (β₂*) as defined above.

In some embodiments, X is chosen from moieties of formulas B, E and O. In some embodiments, X is chosen from moieties of formula A, when n is 1. In some embodiments, X is chosen from moieties of formula J, when m+n is 1 or 2.

In some embodiments, the compound of the present invention is chosen from pharmaceutically acceptable salts of compounds of formula (I).

In some embodiments, the compound of the present invention is chosen from hydrates of compounds of formula (I).

In some embodiments, the compound of the present invention is chosen from solvates of compounds of formula (I).

In some embodiments, the compound of the present invention is chosen from metabolites of compounds of formula (I).

In some embodiments, the compound of the present invention is chosen from prodrugs of compounds of formula (I).

In some embodiments, the compound of the present invention is chosen from isosteres of compounds of formula (I).

Examples of a compound of formula (I) include without limitation:

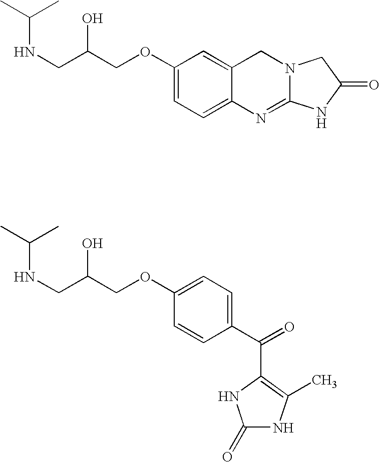

### EXAMPLE 1

### 6-{2-hydroxy-3-[(methylethyl)amino]-propoxy}-4,3a-dihydroimidazolidino[2,1-b]-quinazolin-2-one

### EXAMPLE 2

### 5-[(4-{2-hydroxy-3-[(methylethyl)-aminopropoxy}phenyl)carbonyl-4-methyl-4-imidazolin-2-one

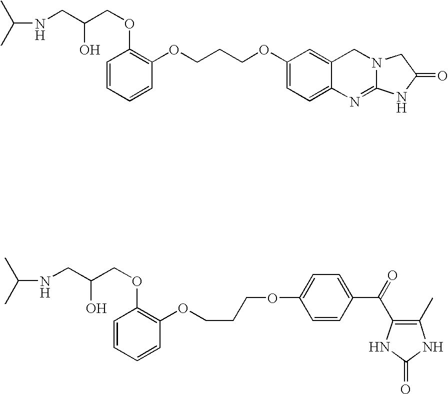

### EXAMPLE 3

### 6-[3-(2-{2-hydroxy-3-[(methylethyl)-amino]propoxy}phenoxy)propoxy]-4,3a-dihydro-imidazolidino[2,1-b]quinazolin-2-one

### EXAMPLE 4

### 5-({4-[3-(2-{2-hydroxy-3-[(methyethyl-aminopropoxy}phenoxy)propoxy]phenyl}carbonyl)-4-methyl-4-imidazolin-2-one

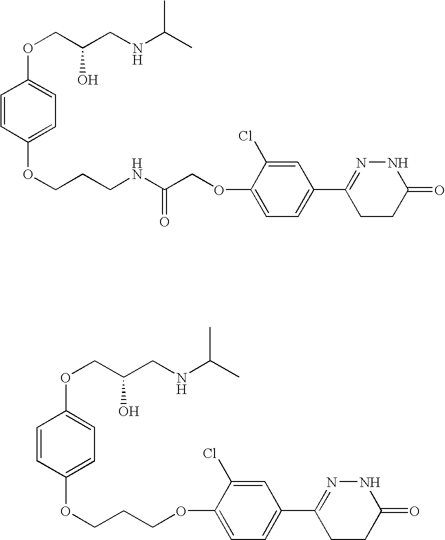

### EXAMPLE 5

### N-[3-(4-{(2S)-2-hydroxy-3-[(methylethyl)-amino]propoxy}phenoxy)propyl]-2-[2-chloro-4-(6-oxo(1,4,5-trihydropyridazin-3-yl))phenoxy]acetamide

### EXAMPLE 6

### N-[3-(4-{(2S)-2-hydroxy-3-[(methylethyl)-amino]propoxy}-phenoxy)propyl]-2-[4-(5-cyano-2-methyl-6-oxo(3-hydropyridyl)phenoxy]acetamide

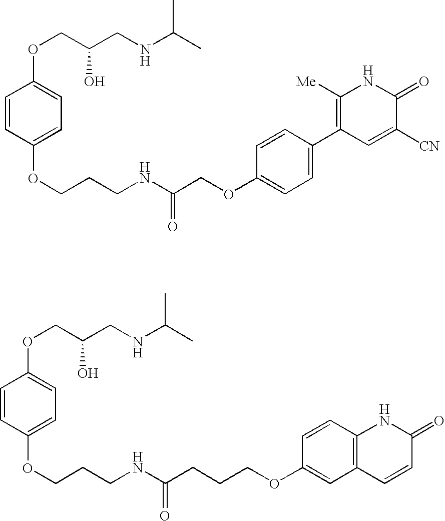

### EXAMPLE 7

### N-[3-(4-{(2S)-2-hydroxy-3-[(methylethyl)amino]propoxy}phenoxy)propyl]-4-(2-oxo(6-hydroquinolyl-oxy))butanamide

### EXAMPLE 8

### 6-{4-[3-(4-(2S)-2-hydroxy-3-[(methylethyl)amino]propoxy}-phenoxy)-propoxy]-3-chlorophenyl}-2,4,5-trihydropyridazin-3-one

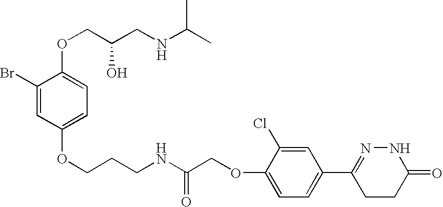

### EXAMPLE 9

### N-[3-(4-{(2S)-2-hydroxy-3-[(methylethyl)amino]propoxy}-3-bromophenoxy)propyl]-2-[2-chloro-4-(6-oxo(1,4,5-trihydropyridazin-3-yl))phenoxy]acetamide

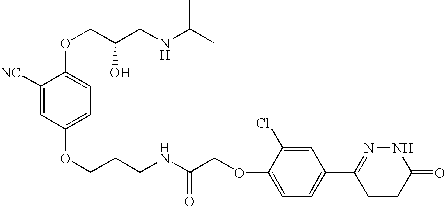

### EXAMPLE 10

### N-[3-(4-{(2S)-2-hydroxy-3-[(methylethyl)amino]propoxy}-3-cyanophenoxy)propyl]-2-[2-chloro-4-(6-oxo(1,4,5-trihydropyridazin-3-yl))phenoxy]acetamide

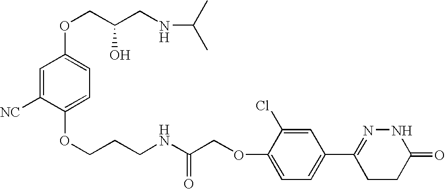

### EXAMPLE 11

### N-[3-(4-{(2S)-2-hydroxy-3-[(methylethyl)amino]propoxy}-2-cyanophenoxy)propyl]-2-[2-chloro-4-(6-oxo(1,4,5-trihydropyridazin-3-yl))phenoxy]acetamide

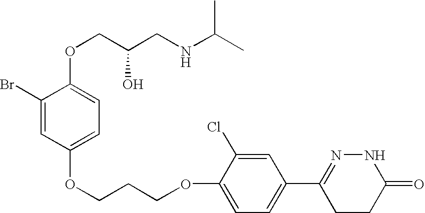

### EXAMPLE 12

### 6-{4-[3-(4-{(2S)-2-hydroxy-3-[(methylethyl)amino]propoxy}-3-bromophenoxy)propoxy]-3-chlorophenyl}-2,4,5-trihydropyridazin-3-one

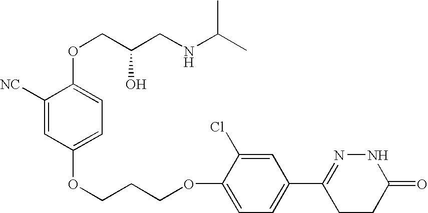

### EXAMPLE 13

### 2-{(2S)-2-hydroxy-3-[(methylethyl)amino]propoxy}-5-{3-[2-chloro-4-(6-oxo(1,4,5-trihydropyridazin-3-yl))phenoxy]propoxy}benzenecarbonitrile

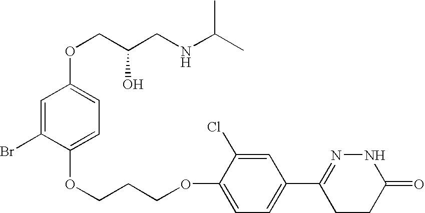

### EXAMPLE 14

### 6-{4-[3-(4-{(2S)-2-hydroxy-3-[(methylethyl)amino]propoxy}-2-bromophenoxy)propoxy]-3-chlorophenyl}-2,4,5-trihydropyridazin-3-one

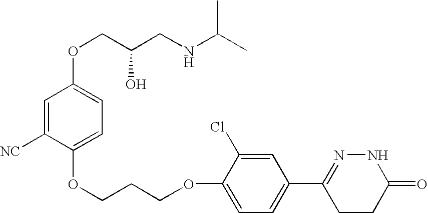

### EXAMPLE 15

### 5-{(2S)-2-hydroxy-3-[(methylethyl)amino]propoxy}-2-{3-[2-chloro-4-(6-oxo(1,4,5-trihydropyridazin-3-yl))phenoxy]propoxy}benzenecarbonitrile

### Pharmaceutical Compositions

This invention further provides a pharmaceutical composition comprising:

(i) an effective amount of a compound of the present invention; and

(ii) a pharmaceutically-acceptable carrier.

In some embodiments, the pharmaceutically-acceptable carrier is chosen from wetting agents, buffering agents, suspending agents, lubricating agents, emulsifiers, disintegrants, absorbents, preservatives, surfactants, colorants, flavorants, sweeteners, and therapeutic agents other than those compounds of the present invention.

In some embodiments, the pharmaceutically-acceptable carrier is chosen from fillers, diluents, excipients, and solvent encapsulating materials. In some embodiments, the pharmaceutically-acceptable carrier is active with respect to the patient. In some embodiments, the pharmaceutically-acceptable carrier are chosen from: (1) sugars, such as lactose, glucose and sucrose; (2) starches, such as corn starch and potato starch; (3) cellulose band its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; and (21) polyesters, polycarbonates and polyanhydrides.

In some embodiments, the pharmaceutically-acceptable carrier is liquid and in others it is solid.

The inventive pharmaceutical composition may be formulated for administration in solid or liquid form, including those adapted for the following: (1) oral administration, for example, drenches (for example, aqueous or non-aqueous solutions or suspensions), tablets, (for example, those targeted for buccal, sublingual, and systemic absorption), boluses, powders, granules, pastes for application to the tongue, hard gelatin capsules, soft gelatin capsules, mouth sprays, emulsions and microemulsions; (2) parenteral administration, for example, by subcutaneous, intramuscular, intravenous or epidural injection as, for example, a sterile solution or suspension, or a sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

### Methods of Use

The present invention further provides a method for regulating calcium homeostasis, comprising administering an effective amount of a compound of the present invention to an animal in need of such regulation.

The present invention further provides a method for treating a disease, disorder or condition in which disregulation of calcium homeostasis is implicated, comprising administering an effective amount of a compound of the present invention to an animal in need of such treatment.

The present invention also provides a method for treating cardiovascular disease, stroke, epilepsy, an ophthalmic disorder or migraine, comprising administering an effective amount of a compound of the present invention to an animal in need of such treatment.

In one embodiment of the present invention, the cardiovascular disease is heart failure, hypertension, SA/AV node disturbance, arrythmia, hypertrophic subaortic stenosis or angina. In another embodiment of the inventive method, the heart failure is chronic heart failure or congestive heart failure.

The present invention further provides a method of inhibiting β-adrenergic receptors and/or inhibiting phosphodiesterase PDE, including PDE3, comprising administering an effective amount of a compound of the present invention to an animal in need of such treatment.

The compound of the present invention may be administered by any means known to an ordinarily skilled artisan. For example, the compound of the present invention may be administered orally, parenterally, by inhalation spray, topically, rectally, nasally, buccally, vaginally, or via an implanted reservoir. The term “parenteral” as used herein includes subcutaneous, intravenous, intramuscular, intraperitoneal, intrathecal, intraventricular, intrastemal, intracranial, and intraosseous injection and infusion techniques. The exact administration protocol will vary depending upon various factors including the age, body weight, general health, sex and diet of the patient; the determination of specific administration procedures would be routine.

The compound of the present invention may be administered by a single dose, multiple discrete doses, or continuous infusion. Pump means, particularly subcutaneous pump means, are useful for continuous infusion.

Dose levels on the order of about 0.001 mg/kg/d to about 10,000 mg/kg/d of compound of the present invention are useful for the inventive method, with preferred levels being about 0.1 mg/kg/d to about 1,000 mg/kg/d, and more preferred levels being about 1 mg/kg/d to about 100 mg/kg/d. The specific dose level for any particular patient will vary depending upon a variety of factors, including the activity and the possible toxicity of the specific compound employed; the age, body weight, general health, sex, and diet of the patient; the time of administration; the rate of excretion; drug combination; the severity of the congestive heart failure, and the form of administration. Typically, in vitro dosage-effect results provide useful guidance on the proper doses for patient administration. Studies in animal models are also helpful. The considerations for determining the proper dose levels are well known in the art and within the skill of a physician.

Any administration regimen well known to an ordinarily skilled artisan for regulating the timing and sequence of drug delivery can be used and repeated as necessary to effect treatment in the inventive method. A further regimen may include pretreatment and/or co-administration with additional therapeutic agents.

The compound of the present invention can be administered alone or in combination with one or more additional therapeutic agent(s) for simultaneous, separate, or sequential use. The additional agent(s) can be any therapeutic agent(s), including without limitation one or more compound(s) of the present invention. The compound of the present invention can be co-administered with one or more therapeutic agent(s) either (i) together in a single formulation, or (ii) separately in individual formulations designed for optimal release rates of their respective active agent.

The compounds of the present invention may be readily made. For example, when m+n is 0 and β and X are directly bonded, the compounds of the present invention may be prepared using standard aromatic chemistry known to those skilled in the art. As shown in general Scheme 1 below, protected aryl hydroxyl precursors of moieties X (P may be e.g., acetyl, benzyl, alkylsilyl, or other appropriate protecting group and Q-T are chosen to reach a particular moiety X) may be deprotected and then may be reacted with epichlorohydrin to yield epoxide intermediates which may be reacted with amines to yield the final products.

Furthermore, such a scheme could readily be adapted to link Ar to β or to link Ar to L or to link Ar to X.

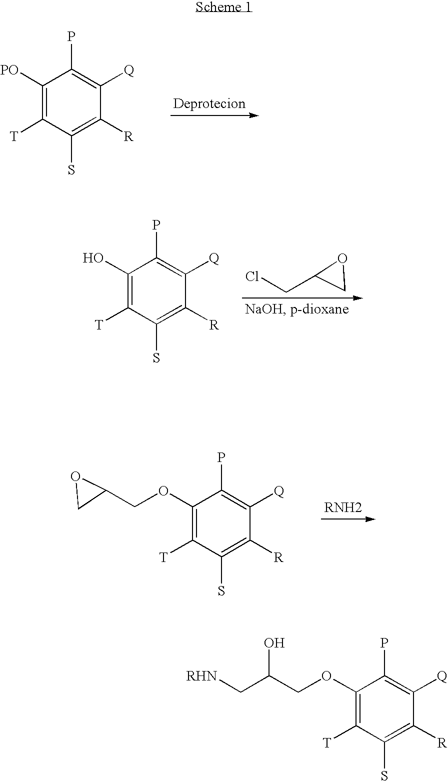

In cases m is 1, wherein X and β or X and Ar are connected by a linker of one or more atoms, the linker may be attached to β, Ar, or X, and the intermediate moiety β-L or X-L or L-Ar may then be linked to X or Ar/β or β/X, respectively, to form A-(Ar)ₙ-L-X.

For example, a general method for preparing β-(Ar)ₙ-L may proceed as follows. Protected phenols of the type depicted below in general Scheme 2 may be reacted with suitably protected linker chains L. “J” in the scheme may be any of various species known to those skilled in the art which can be reacted with a hydroxyl group. For example, J may be a bromine atom, which can be displaced by reaction with the anion of the phenol, or J may be an alcohol group which can be reacted with the phenol under Mitsunobu reaction conditions. P′ may be a suitable protecting group which can be removed under different condition than those which cleave P. The partially deprotected compound may be reacted with a precursor of moiety X or a precursor of Ar, as described in general Scheme 4, before attaching the remaining β constituent. Such a scheme could be readily adapted to link L to Ar or to link β-L to Ar by one of ordinary skill in the art.

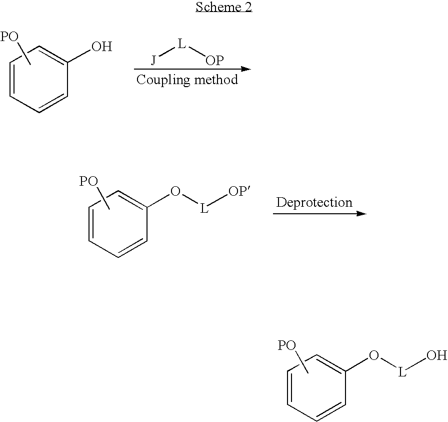

In addition, a general method for preparation of X—(Ar)ₙ-L is analogous to the method for β-(Ar)ₙ-L may proceed as follows. Precursors of moieties X with a hydroxyl group on one of the rings may be reacted with a protected linker group as described in Scheme 2 above and may be subsequently deprotected. Such a scheme could be readily adapted to link X to Ar or to link X to L-(Ar)ₙ-β or to link X to Ar-β by one of ordinary skill in the art.

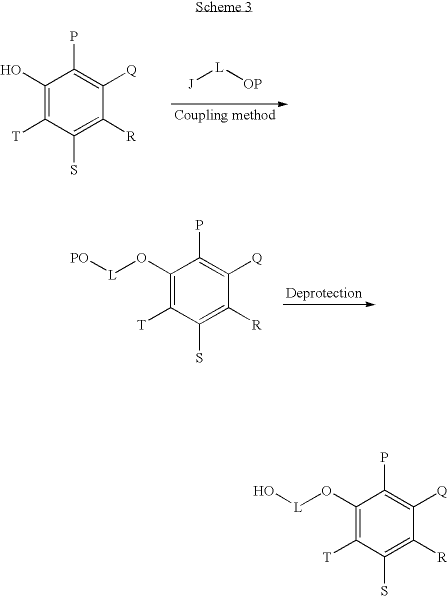

General method for reacting A-L or X-L with X or A to make A-L-X may proceed as follows. A resultant compound from general Scheme 2 may be reacted with an aryl hydroxyl precursor of moiety X via standard Mitsunobu chemistry as shown below in Scheme 4. Following deprotection of the remaining hydroxyl group, sequential reaction with epichlorohydrin and a substituted amine may deliver the final product.

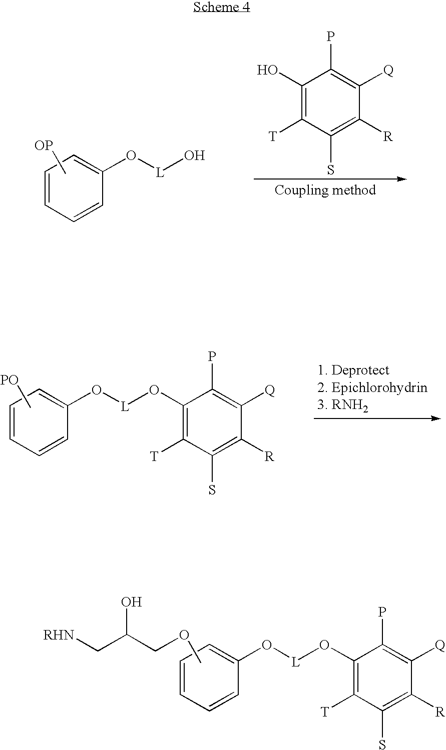

Indeed, general Schemes 1-4 could be readily adapted to make X-(L)ₘ-(Ar)ₙ-β by one of ordinary skill in the art.

A compound from general Scheme 3 may similarly be reacted with a protected phenol as shown below, and the coupling product may be converted to the final compound by the same deprotection/reaction with epichlorohydrin/reaction with RNH₂ sequence as previously described.

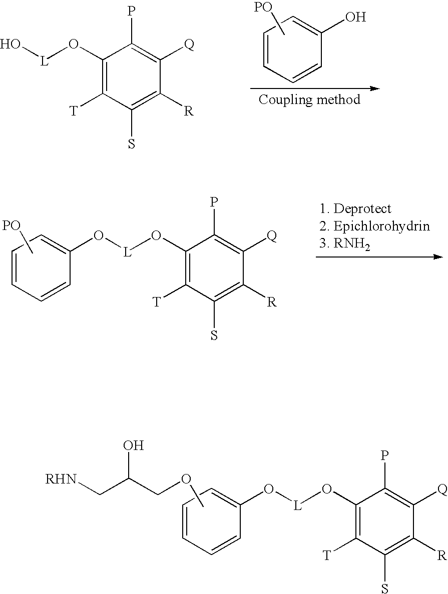

### EXAMPLES

### Example 1

### 6-{2-hydroxy-3-[(methylethyl)amino]propoxy}-4,3a-dihydroimidazolidino[2,1-b]quinazolin-2-one is Synthesized According to the Method of Scheme I

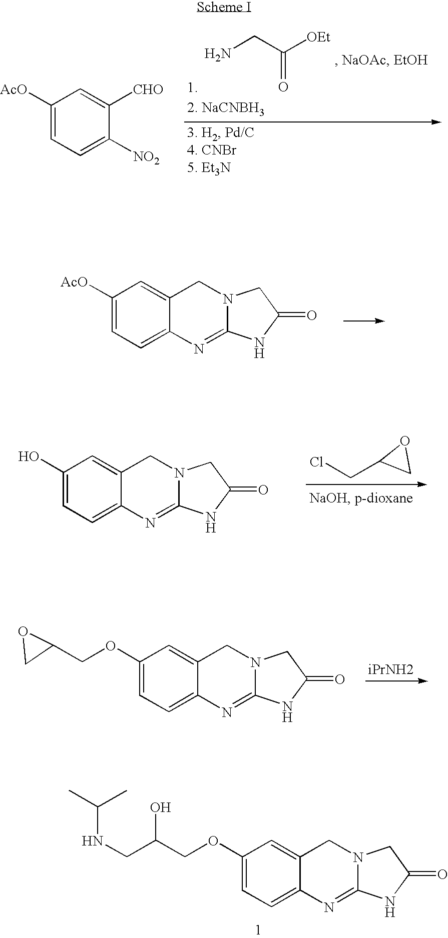

2-oxo-4,3a-dihydroimidazolidino[2,1-b]quinazolin-6-yl acetate: 3-formyl-4-nitrophenyl acetate (10 mmol) is added to a solution prepared from glycine ethyl ester hydrochloride (3.0 g, 24 mmol) and anhydrous sodium acetate (820 mg, 10 mmol) in methanol (80 mL). After stirring the thick mixture for 15 minutes, sodium cyanoborohydride (380 mg, 6 mmol) is added, resulting in dissolution of the precipitate. After stirring for an hour, the solvent is evaporated and the residue is partitioned between ethyl acetate (50 mL) and saturated aqueous NaHCO₃ (50 mL). The layers are separated and the aqueous phase is extracted with additional ethyl acetate. The combined organic fractions are washed with saturated aqueous NaHCO₃ and brine, dried over magnesium sulfate, and concentrated in vacuo. The crude residue is purified by silica gel chromatography to furnish the benzylamine intermediate, which is dissolved in 20 mL of ethanol and hydrogenated at 60 psi over 10% Pd—C overnight. After removing the catalyst by filtration, a solution of cyanogen bromide (760 mg; 7.1 mmol) in 5 mL of ethanol is added to the filtrate. After stirring overnight, the mixture is treated with triethylamine (1.1 mL, 7.8 mmol) and stirring is continued overnight again. The formed precipitate is collected by filtration, washed repeatedly with water and ethanol-ether, and dried to provide the title compound.

6-hydroxy-4,3a-dihydroimidazolidino[2,1-b]quinazolin-2-one: The above compound is suspended in 10 mL of methanol and treated with 2 mL of a 2.5 M solution of NaOH. After stirring for 1 hour, the precipitate is collected by filtration, washed with acetone, and dried under vacuum to furnish the phenol as a solid.

6-(oxiran-2-ylmethoxy)-4,3a-dihydroimidazolidino[2,1-b]quinazolin-2-one: 6-Hydroxy-4,3a-dihydroimidazolidino[2,1-b]quinazolin-2-one (3.8 mmol) is added to a solution of NaOH (150 mg; 3.8 mmol) in 5 mL of H₂O. Epichlorohydrin (2.5 mL, 32 mmol) and p-dioxane are added, and the reaction is stirred for 24 hours under inert atmosphere. The reaction mixture is extracted with methylene chloride, and the organic phase is washed with brine and water, dried, and concentrated to deliver the crude product as a brown oil. The crude material is purified on a silica gel column eluting with 25% hexane in ethyl acetate to deliver the pure product as a solid.

6-{2-hydroxy-3-[(methylethyl)amino]propoxy}-4,3a-dihydroimidazolidino[2,1-b]quinazolin-2-one: The epoxide above (2.7 mmol) and isopropylamine (3.8 mmol) are dissolved in methanol (5 mL) and stirred together for 36 hrs. The solvent is removed under vacuum and the crude residue is applied to a silica gel column, eluting with 5% methanol in CH₂Cl₂, to deliver the compound of example 1.

### Example 2

### 5-[(4-{2-hydroxy-3-[(methylethyl)amino]propoxy}phenyl)carbonyl]-4-methyl-4-imidazolin-2-one is Synthesized According to the Method of Scheme II

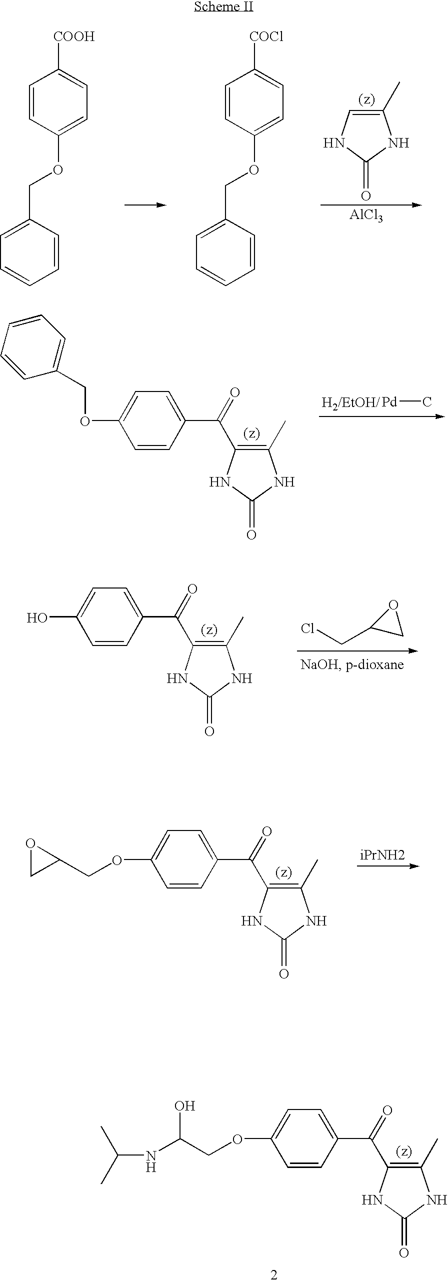

4-methyl-5-{[4-(phenylmethoxy)phenyl]carbonyl}-4-imidazolin-2-one: The potassium salt of 4-(phenylmethoxy)benzoic acid (56 mmol) is suspended in 150 mL of CH₂Cl₂, cooled in an ice-bath, and treated with 7.50 g (60 mmol) of oxalyl chloride added dropwise. Following the completion of the addition, the mixture is refluxed for 30 minutes, cooled, and filtered. The filtrate was added dropwise to a stirred mixture of 4-methyl-4-imidazolin-2-one (56 mmol, prepared by the method of Duschinsky and Dolan, J. Am. Chem. Soc. 1945, 67, 2079) and anhydrous aluminum chloride (112 mmol) in 50 mL of nitrobenzene. The resulting mixture is stirred at 65° C. for 6 hours and then poured over ice. The precipitate formed is collected by filtration, washed with ether and water, and recrystallized from ethanol/water to deliver the product.

5-[(4-hydroxyphenyl)carbonyl]-4-methyl-4-imidazolin-2-one: The benzyl protected compound (15 mmol) is dissolved in ethanol, treated with a catalytic amount of 10% palladium on carbon, and hydrogenated at 50 psi overnight. The catalyst is removed by filtration and the solvent was removed in vacuo to yield the crude product as an oil, which is used directly for the next step.

4-methyl-5-{[4-(oxiran-2-ylmethoxy)phenyl]carbonyl}-4-imidazolin-2-one: The phenol (3.5 mmol) is added to a solution of NaOH (150 mg; 3.8 mmol) in 5 mL of H₂O. Epichlorohydrin (2.5 mL, 32 mmol) and p-dioxane are added, and the reaction is stirred for 24 hours under inert atmosphere. The reaction mixture is extracted with methylene chloride, and the organic phase is washed with brine and water, dried, and concentrated to deliver the crude product as an oil. The crude material is purified on a silica gel column eluting with 20% hexane in ethyl acetate to deliver the pure product.

5-[(4-{2-hydroxy-2-[(methylethyl)amino]ethoxy}phenyl)carbonyl]-4-methyl-4-imidazolin-2-one: The epoxide above (2 mmol) and isopropylamine (4 mmol) are dissolved in methanol (5 mL) and stirred together for 36 hrs. The solvent is removed under vacuum and the crude residue is applied to a silica gel column, eluting with 10% methanol in CH₂Cl₂, to deliver the compound of example 2.

### Example 3

### 6-[3-(2-{2-hydroxy-3-[(methylethyl)amino]propoxy}phenoxy)propoxy]-4,3a-dihydro-imidazolidino[2,1-b]quinazolin-2-one is Prepared According to the Method of Scheme III

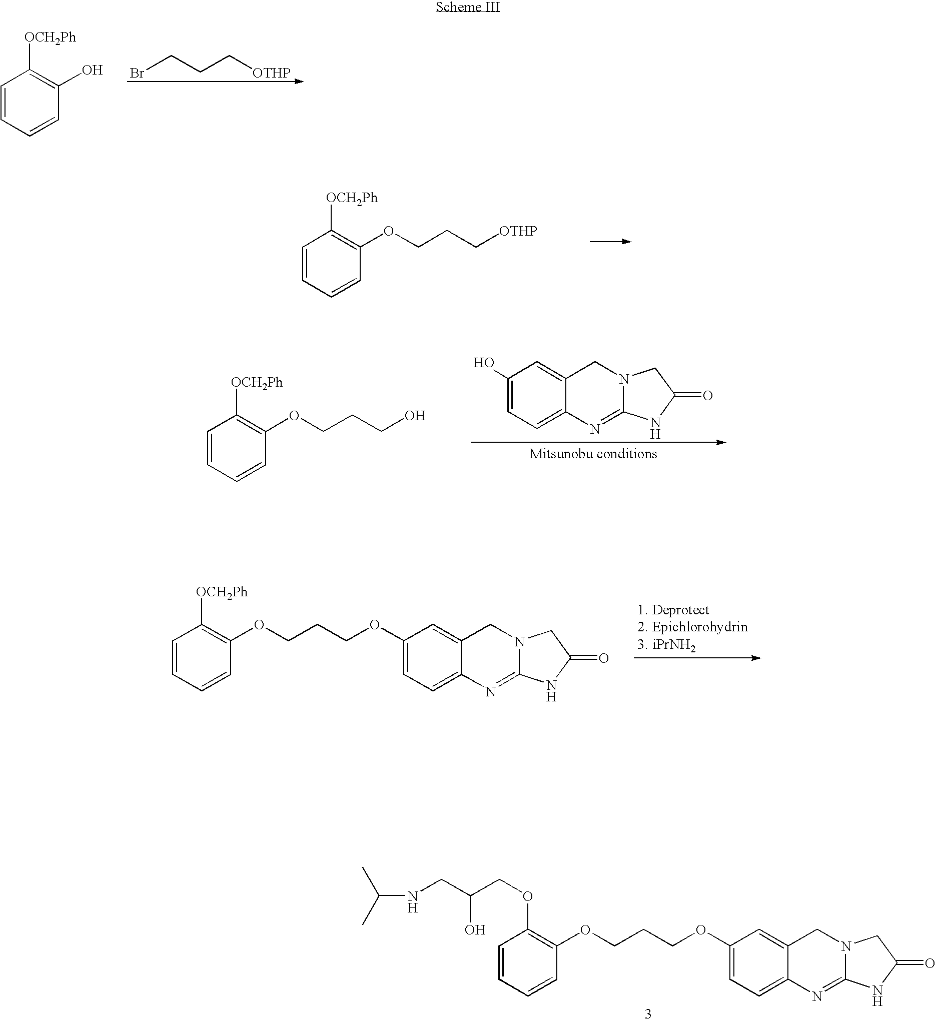

1-(3-perhydro-2H-pyran-2-yloxypropoxy)-2-(phenylmethoxybenzene: Sodium hydride (10 mmol) is added to a solution of 2-(phenylmethoxy)phenol (9 mmol) in 50 mL of dry ether, and subsequently treated with 12 mmol of 3-bromo-1-perhydro-2H-pyran-2-yloxypropane in 10 mL of ether. The mixture is stirred at 70° C. for 5 hours, then quenched by the addition of 2 mL of methanol followed by partitioning between ethyl acetate and water. The organic phase is washed with brine, dried, concentrated, and the crude residue is purified on a silica gel column, eluting with 5% ethyl acetate in hexane, to obtain the product as a clear oil.

3-[2-(phenylmethoxy)phenoxy]propan-1-ol: The tetrahydropyranyl-protected alcohol (10 mmol) is dissolved in methylene chloride (20 mL) and treated with 2 mmol of para-toluenesulfonic acid. After stirring at room temperature overnight, the reaction mixture is partitioned between methylene chloride and brine, concentrated, and the crude residue is purified on a silica gel column, eluting with 25% ethyl acetate in hexane, to obtain the product as a clear oil.

6-{3-[2-(phenylmethoxy)phenoxy]propoxy}-4,3a-dihydroimidazolidino[2,1-b]quinazolin-2-one: A mixture of 3-[2-(phenylmethoxy)phenoxy]propan-1-ol and 6-hydroxy-4,3a-dihydroimidazolidino[2,1-b]quinazolin-2-one (prepared as in Scheme I) are coupled using diethyl azodicarboxylate and triethylphosphine according to the method of Mitsunobu (Bull. Chem. Soc. Jpn., 1979, 52, 1191-1196).

6-[3-(2-hydroxyphenoxy)propoxy]-4,3 a-dihydroimidazolidino[2,1-b]quinazolin-2-one: The benzyl protected compound (11 mmol) is dissolved in ethanol, treated with a catalytic amount of 10% palladium on carbon, and hydrogenated at 50 psi overnight. The catalyst is removed by filtration and the solvent was removed in vacuo to yield the crude product as an oil, which is used directly for the next step.

6-{3-[2-(cyclopropylmethoxy)phenoxy]propoxy}-4,3a-dihydroimidazolidino[2,1-b]quinazolin-2-one: The phenol (4 mmol) is added to a solution of NaOH (150 mg; 4.4 mmol) in 5 mL of H₂O. Epichlorohydrin (2.8 mL, 35 mmol) and p-dioxane are added, and the reaction is stirred for 24 hours under inert atmosphere. The reaction mixture is extracted with methylene chloride, and the organic phase is washed with brine and water, dried, and concentrated to deliver the crude product as an oil. The crude material is purified on a silica gel column eluting with 20% hexane in ethyl acetate to deliver the pure product.

6-[3-(2-{2-hydroxy-3-[(methylethyl)amino]propoxy}phenoxy)propoxy]-4,3a-dihydro-imidazolidino[2,1-b]quinazolin-2-one: The epoxide above (2.2 mmol) and isopropylamine (4.4 mmol) are dissolved in methanol (5 mL) and stirred together for 36 hrs. The solvent is removed under vacuum and the crude residue is applied to a silica gel column, eluting with 10% methanol in CH₂Cl₂, to deliver the compound of example 3.

### Example 4

### 5-({4-[3-(2-{2-hydroxy-3-[(methylethyl)amino]propoxy}phenoxy)propoxy]phenyl}carbonyl)-4-methyl-4-imidazolin-2-one is Prepared According to the Method of Scheme IV

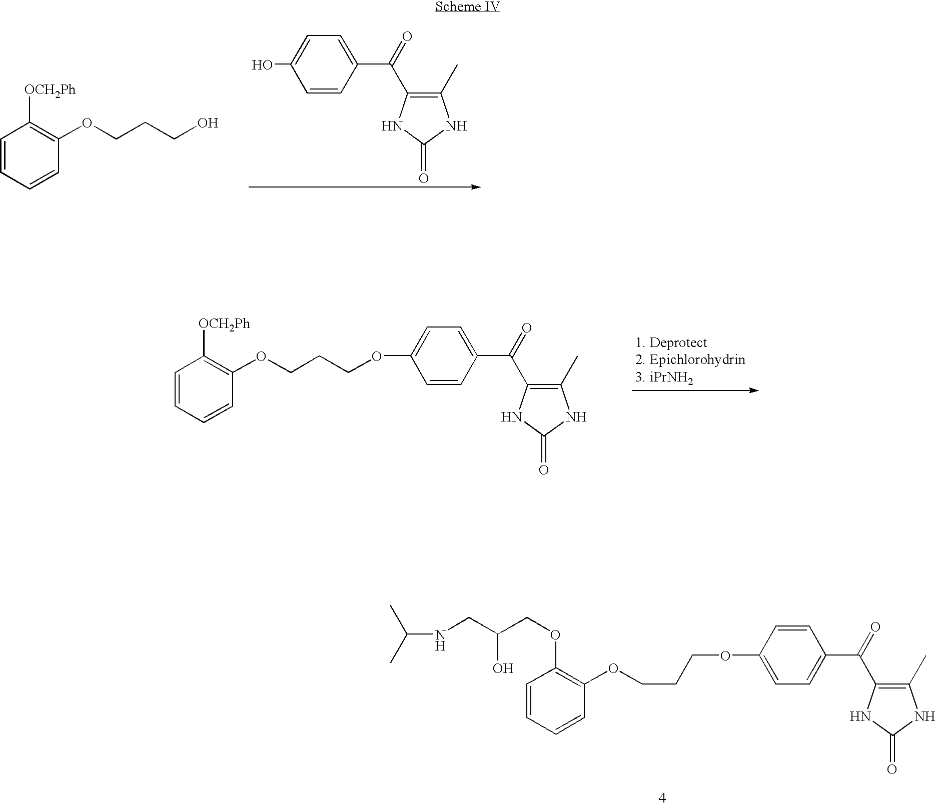

4-methyl-5-[(4-{3-[2-(phenylmethoxy)phenoxy]propoxy}phenyl)carbonyl]-4-imidazolin-2-one: 3-[2-(phenylmethoxy)phenoxy]propan-1-ol ( ) and 5-[(4-hydroxyphenyl)carbonyl]-4-methyl-4-imidazolin-2-one are coupled using diethyl azodicarboxylate and triethylphosphine according to the method of Mitsunobu (Bull. Chem. Soc. Jpn., 1979, 52, 1191-1196).

5-({4-[3-(2-{2-hydroxy-3-[(methylethyl)amino]propoxy}phenoxy)propoxy]phenyl}carbonyl)-4-methyl-4-imidazolin-2-one (4) is prepared from the product of the previous step by the same sequence of reactions (deprotection, reaction with epichlorohydrin, and subsequent reaction of the epoxide with isopropylamine sequence as described in the previous schemes, as described in Scheme III, to yield the compound of Example 4.

### Example 5

### N-[3-(4-{(2S)-2-hydroxy-3-[(methylethyl)amino]propoxy}phenoxy)propyl]-2-[2-chloro-4-(6-oxo(1,4,5-trihydropyridazin-3-yl))phenoxy]acetamide was Prepared According to the Method of Scheme V

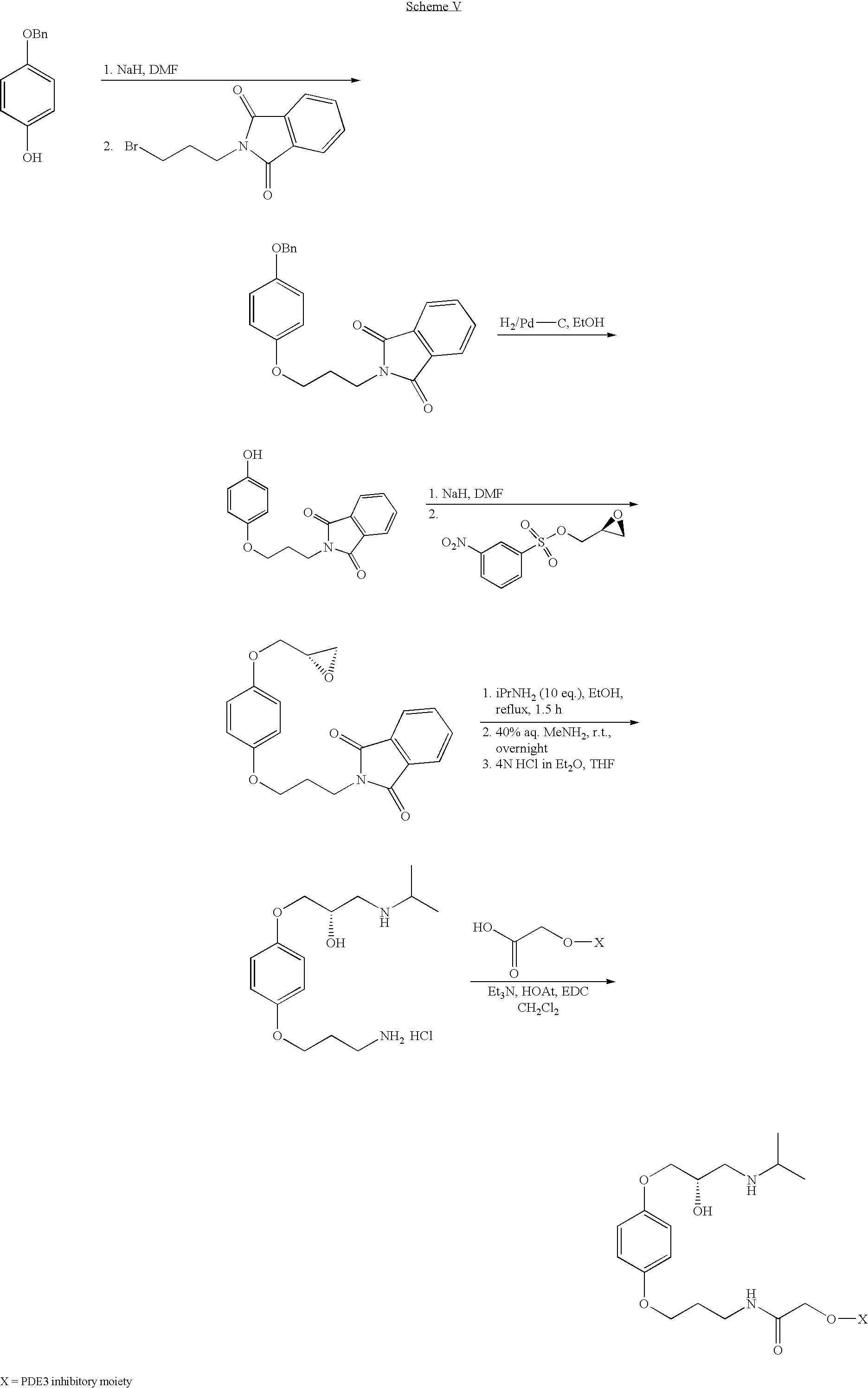

2-[3-(4-Hydroxy-phenoxy)-propyl]-isoindole-1,3-dione: To a stirred solution of 2-[3-(4-benzyloxy-phenoxy)-propyl]-isoindole-1,3-dione (1.25 g, 3.23 mmol) in ethanol/ethyl acetate (2:1) (60 mL) was added palladium on activated carbon (10 wt % Pd, wet Degussa type with 50 wt % water, 315 mg, 0.148 mmol). The reaction mixture was stirred under an atmosphere of hydrogen (1.5 atm) for 16 hours at ambient temperature and then filtered through a pad of Celite®. The filtrate was evaporated to dryness and the residue was purified by flash chromatography over silica gel (50 g) using dichloromethane/methanol (99:1) as eluent. Fractions with R_{f}=0.33 (DCM/MeOH 98:2) were combined and concentrated under reduced pressure. The residue was recrystallised from ethyl acetate to give 2-[3-(4-hydroxy-phenoxy)-propyl]-isoindole-1,3-dione as colorless plates (730 mg, 76% yield, 99% pure by LC-MS and ¹H-NMR). ¹H NMR (400 MHz; CDCl₃): δ 8.13 (m, 2H); 7.69 (m, 2H); 6.62-6.60 (m, 4H); 3.94 (m, 2H); 3.63 (m, 2H); 2.04 (m 2H).

2-[3-(4-Oxiranylmethoxy-phenoxy)-propyl]-isoindole-1,3-dione: To a stirred suspension of sodium hydride (60% dispersion in mineral oil, 108 mg, 2.70 mmol) in N,N-dimethylformamide (6 mL) under nitrogen at 0° C. was added 2-[3-(4-hydroxy-phenoxy)-propyl]-isoindole-1,3-dione (730 mg, 2.45 mmol) and the reaction mixture was stirred for 20 minutes at ambient temperature. A solution of 3-nitro-benzenesulfonic acid oxiranyl-methyl ester (700 mg, 2.70 mmol) in N,N-dimethylformamide (6 mL) was added at 0° C. The mixture was stirred at ambient temperature for 16 hours, then poured onto a mixture of ice and saturated aqueous ammonium chloride solution (50 mL) and extracted with ethyl acetate (4×25 mL). The combined organic extracts were washed with saturated brine (2×25 mL), dried (Na₂SO₄) and concentrated under reduced pressure. The residue was dissolved in dichloromethane, adsorbed onto silica, evaporated to dryness and the residue dry-loaded onto a silica gel column (50 g). Purification by column chromatography was carried out using a gradient of neat dichloromethane to dichloromethane/ethyl acetate (9:1) as eluent. Fractions with R_{f}=0.54 (DCM) were combined and evaporated to dryness under reduced pressure to give 2-[3-(4-oxiranylmethoxy-phenoxy)-propyl]-isoindole-1,3-dione as a colorless solid (460 mg, 53% yield, 95% pure by LC-MS and ¹H-NMR). ¹H NMR (400 MHz; CDCl₃): δ 8.13 (m, 2H); 7.69 (m, 2H); 6.66 (m, 4H); 4.07 (m, 2H); 3.94 (m, 1H); 3.63 (m, 2H); 3.04 (m, 1H); 2.50 (m, 2H); 2.04 (m, 2H).

1-[4-(3-Amino-propoxy)-phenoxy]-3-isopropylamino-propan-2-ol via 2-{3-[4-(2-Hydroxy-3-isopropylamino-propoxy)-phenoxy]-propyl}-isoindole-1,3-dione: To a stirred solution of 2-[3-(4-oxiranylmethoxy-phenoxy)-propyl]-isoindole-1,3-dione (460 mg, 1.30 mmol) in ethanol (20 mL) was added iso-propylamine (1.11 mL, 13.0 mmol). The reaction mixture was heated to reflux, then stirred at this temperature for 3 hours, and then concentrated under reduced pressure to give crude 2-{3-[4-(2-hydroxy-3-isopropylamino-propoxy)-phenoxy]-propyl}-isoindole-1,3-dione. The residue was dissolved in methylamine (40 wt % in water, 20 mL), stirred at ambient temperature for 16 hours, then diluted with H₂O (20 mL) and brine (20 mL), and extracted with dichloromethane (4×20 mL). The combined organic layers were washed with brine (2×10 mL), dried (Na₂SO₄) and concentrated under reduced pressure to give crude 1-[4-(3-amino-propoxy)-phenoxy]-3-isopropylamino-propan-2-ol as light yellow oil (355 mg, 96% yield, 90% pure by LC-MS and ¹H-NMR), which was used without further purification. ¹H NMR (400 MHz; CDCl₃): δ 6.68 (m, 4H); 4.09 (m, 2H); 3.96 (m, 1H); 3.94 (m, 2H); 2.97 (m, 1H); 2.70 (m, 2H); 2.65 (m, 2H); 1.97 (m, 2H); 1.05 (d, 6H total).

2-[2-Chloro-4-(6-oxo-1,4,5,6-tetrahydro-pyridazin-3-yl)-phenoxy]-N-{3-[4-(2-hydroxy-3-isopropylamino-propoxy)phenoxy]propyl}acetamide: To a stirred solution of [2-chloro-4-(6-oxo-1,4,5,6-tetrahydro-pyridazin-3-yl)-phenoxy]-acetic acid (126 mg, 0.446 mmol), 1-(3-dimethylaminopropyl)-3-ethyl carbodiimide hydrochloride (EDC.HCl, 85.4 mg, 0.446 mmol) and 7-hydroxyazabenzotriazole (HOAt, 60.7 mg, 0.446 mmol) in N,N-dimethylformamide (4 mL) under N₂ was added a solution of crude 1-[4-(3-amino-propoxy)-phenoxy]-3-isopropylamino-propan-2-ol (140 mg, 0.496 mmol) in N,N-dimethylformamide (2 mL), and the mixture was stirred at ambient temperature for 3 hours. The reaction mixture was poured into saturated brine (40 mL), made strongly alkaline (pH 11-12) with aqueous sodium hydroxide solution (2 N), and extracted with ethyl acetate (4×20 mL). The combined organic layers were washed with saturated brine (2×20 mL), dried (Na₂SO₄) and concentrated under reduced pressure. The residue was dry-loaded and purified by column chromatography on silica gel (4 g) using dichloromethane/methanol (9:1) as eluent. Fractions with R_{f}=0.04 were combined and evaporated to dryness under reduced pressure to give 2-[2-chloro-4-(6-oxo-1,4,5,6-tetrahydro-pyridazin-3-yl)-phenoxy]-N-{3-[4-(2-hydroxy-3-isopropylaminopropoxy)-phenoxy]propyl}acetamide as an off-white solid (136 mg, 56% yield, 97% pure by LC-MS and ¹H-NMR). ¹H NMR (400 MHz; CDCl₃): δ 7.51 (d, 1H); 7.41 (dd, 1H); 6.69 (dd, 1H); 6.66 (m, 4H total); 4.83 (s, 2H); 4.09 (d, 1H); 3.96 (m, 1H); 3.94 (m, 2H); 3.20 (m, 2H); 2.97 (dq, 1H); 2.70 (m, 1H); 2.21 (m, 2H); 1.97 (m, 2H); 1.62 (m, 2H); 1.05 (d, 6H total).

The required PDE3 inhibitor fragment, [2-chloro-4-(6-oxo-1,4,5,6-tetrahydro-pyridazin-3-yl)-phenoxy]-acetic acid, was synthesized as described in Scheme V-a:

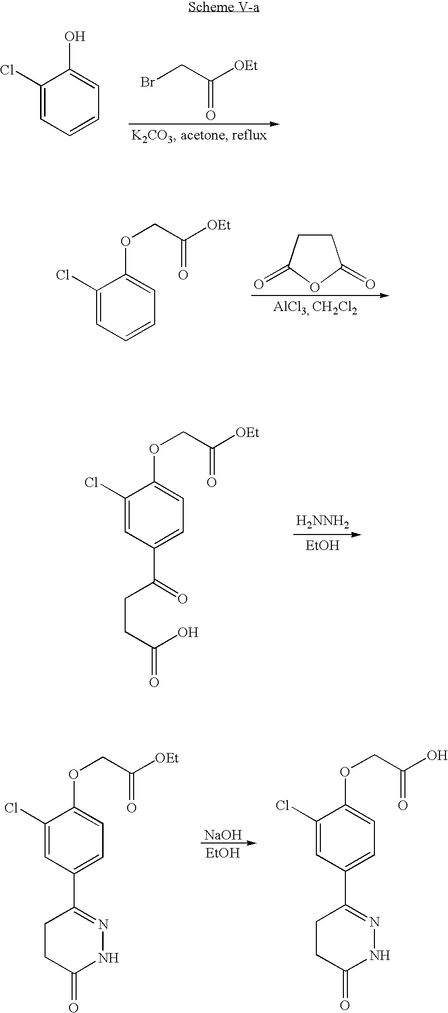

Ethyl 2-chlorophenoxyacetate: To a stirred solution of 2-chlorophenol (20.0 g, 156 mmol) in acetone (300 mL) under nitrogen at ambient temperature were added potassium carbonate (23.7 g, 171 mmol) and ethyl bromoacetate (7, 26.0 g, 156 mmol). The reaction mixture was then heated to reflux and stirred at this temperature under nitrogen for 7 hours. After cooling to ambient temperature, the reaction mixture was filtered to remove insolubles. The filtrate was then concentrated under reduced pressure to give the product as highly viscous, light yellow oil (32.0 g, 95% yield, 95% pure by LCMS and ¹H NMR), ¹H NMR (400 MHz; CDCl₃): δ 7.16 (m, 1H); 7.03 (m, 1H); 6.76 (m, 1H); 6.71 (m, 1H); 4.90 (s, 2H); 4.12 (q, 2H); 1.33 (t, 3H).

4-[3-Chloro-4-(ethoxycarbonylmethoxyphenyl]-4-oxobutyric acid: To a stirred solution of ethyl 2-chlorophenoxyacetate (32.0 g, 149 mmol) in dichloromethane (75 mL) at ambient temperature under nitrogen was added succinic anhydride (22.4 g, 224 mmol). The reaction mixture was cooled in ice-water and to this was added portion wise aluminum trichloride (59.6 g, 447 mmol), whilst maintaining the temperature below 20° C. The reaction mixture was then allowed to stir at ambient temperature for 20 minutes and was then heated to reflux and stirred at this temperature for 3 hours. The reaction mixture was allowed to cool to ambient temperature, then poured into a mixture of ice, water (200 ml) and HCl (10 N, 100 ml). The two phase system was separated and the aqueous layer was extracted with ethyl acetate (5×100 mL). All organic layers were then combined and washed with water (2×100 mL), dried over Na₂SO₄, and concentrated under reduced pressure to give an orange oily solid. Hexane (300 mL) was added, and after standing at ambient temperature for 1 hour, the precipitate was filtered off and re-crystallized from ethyl acetate/hexane to give the diketo compound as a light yellow powder (21.5 g, 46% yield, 98% pure by LCMS and ¹H NMR), ¹H NMR (400 MHz; CDCl₃): δ 7.79 (m, 1H); 7.66 (m, 1H); 6.79 (m, 1H); 4.90 (s, 2H); 4.12 (q, 2H); 2.82 (m, 2H); 2.42 (m, 2H); 1.30 (t, 3H).

6-[3-Chloro-4-(ethoxycarbonylmethoxy)phenyl]-4,5-dihydro-3(2H)-pyridazinone: To a stirred suspension of 4-[3-chloro-4-(ethoxycarbonylmethoxy)phenyl]-4-oxobutyric acid (21.5 g, 69.2 mmol) in ethanol (200 mL) at 0° C. was added a solution of hydrazine monohydrate (3.4 mL, 69.2 mmol) in ethanol (20 mL). The reaction mixture was then allowed to warm to ambient temperature and stirred at this temperature for 15 minutes before being heated to reflux and stirred at this temperature for 3 hours. Ethyl acetate (40 mL) was added to the hot solution and the mixture was allowed to cool to ambient temperature. The precipitate which formed was filtered off and washed with water (2×100 mL) and cold ethanol (2×100 mL), then dried with suction, then under high vacuum to give the pyridazinone as light yellow powder (17.6 g, 82% yield, 99% pure by LCMS and ¹H NMR), ¹H NMR (400 MHz; CDCl₃): δ 7.52 (m, 1H); 7.41 (m, 1H); 6.70 (m, 1H); 4.90 (s, 2H); 4.12 (q, 2H); 2.22 (m, 2H); 1.62 (m, 2H); 1.30 (q, 3H).

Pyridazinone carboxylic acid (6-{4-[3-carboxymethoxy]-3-chlorophenyl}-4,5-dihydro-3(2H)-pyridazinone): To a stirred suspension of 6-[3-chloro-4-(ethoxycarbonyl-methoxy)phenyl]-4,5-dihydro-3(2H)-pyridazinone (17.6 g, 56.6 mmol) in ethanol (150 mL) at ambient temperature were added water (150 mL) and sodium hydroxide (9.10 g, 227 mmol). The reaction mixture was then heated to 80° C. and stirred at this temperature for 2.5 hours. The solution was allowed to cool until precipitation occurred, then the suspension was acidified to pH 1-2 with HCl (2 N, 100 mL) with stirring. After standing at ambient temperature for 1 hour, the precipitate was filtered off and washed with water (2×100 mL) and ethanol (2×100 mL). The solid was dried under high vacuum at 45° C. to give 6-{4-[3-carboxymethoxy]-3-chlorophenyl}-4,5-dihydro-3(2H)-pyridazinone as a light yellow powder (13.4 g, 84% yield, 99% pure by LCMS and ¹H NMR), ¹H NMR (400 MHz; CDCl₃): δ 7.52 (m, 1H); 7.44 (m, lH); 6.72 (m, 1H); 4.88 (s, 2H); 2.21 (m, 2H); 1.61 (m, 2H).

Using the procedure of Scheme V-a, different halo alkanoic acids may be utilized to obtain PDE inhibitor fragments with varying chain lengths.

### Example 6

2-[4-(5-Cyano-2-methyl-6-oxo-1,6-dihydro-pyridin-3-yl)-phenoxy]-N-{3-[4-(2-hydroxy-3-isopropylaminopropoxy)phenoxy]propyl }acetamide was synthesized using the same procedure as was used for Example 5, starting from [4-(5-cyano-2-methyl-6-oxo-1,6-dihydro-pyridin-3-yl)-phenoxy]-acetic acid (127 mg, 0.446 mmol). 2-[4-(5-Cyano-2-methyl-6-oxo-1,6-dihydro-pyridin-3-yl)-phenoxy]-N-{3-[4-(2-hydroxy-3-iso-propylamino-propoxy)-phenoxy]-propyl}-acetamide (Example 6) was isolated as off-white solid (95 mg, 39% yield, 93% pure by LC-MS and ¹H-NMR). ¹H NMR (400 MHz; CDCl₃): δ 7.70 (s, 1H); 7.19 (m, 2H); 6.72 (m, 2H); 6.66 (m, 4H); 4.83 (s, 2H); 4.09 (m, 2H); 3.96 (m, 1H); 3.94 (m, 2H); 3.20 (m, 2H); 2.97 (m, 1H); 1.71 (s, 3H); 1.05 (d, 6H total).

The required PDE3 inhibitor fragment, 2-[4-(5-cyano-2-methyl-6-oxo-3-hydropyridyl)phenoxy]acetic acid, was prepared according to Scheme V-b.

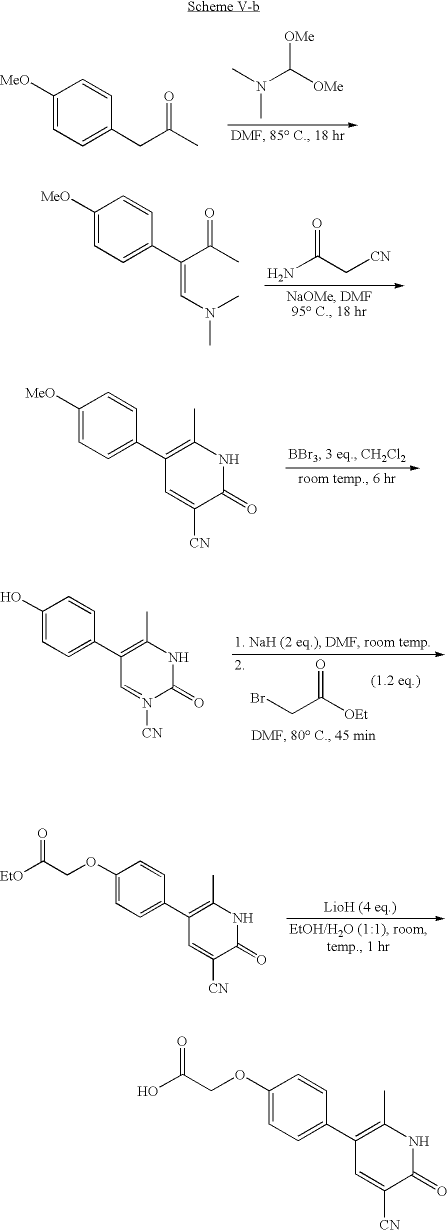

4-Dimethylamino-3-(4-methoxy-phenyl)-but-3-en-2-one: To a stirred solution of 1-(4-methoxy-phenyl)-propan-2-one (8.37 g, 51.0 mmol) in N,N-dimethylformamide (200 mL) was added dimethoxymethyl-dimethyl-amine (27 mL, 203 mmol). The reaction mixture was then stirred for 18 hours at 85° C., allowed to cool to ambient temperature and excess solvent and reagents were removed under reduced pressure to give crude 4-dimethylamino-3-(4-methoxyphenyl)-but-3-en-2-one as yellow oil which was used in the following step without further purification.

5-(4-Methoxy-phenyl)-6-methyl-2-oxo-1,2-dihydropyridine-3-carbonitrile: To a stirred solution of sodium hydride (60% dispersion in mineral oil, 4.5 g, 112 mmol) in N,N-dimethylformamide (100 mL) was added dropwise at 0° C. a solution of crude 4-dimethylamino-3-(4-methoxyphenyl)-but-3-en-2-one from the previous step, 2-cyano-acetamide (4.75 g, 56.5 mmol) and methanol (4.54 mL, 112 mmol) in N,N-dimethylformamide (50 mL). The reaction mixture was stirred at ambient temperature for 15 minutes and then at 95° C. for 18 hours. After cooling to ambient temperature most of the solvent was removed under reduced pressure. The residue was hydrolysed with saturated aqueous ammonium chloride solution (100 mL). The precipitated solid was collected by filtration with suction, rinsed with water and diethyl ether, and dried under vacuum to give 5-(4-methoxy-phenyl)-6-methyl-2-oxo-1,2-dihydro-pyridine-3-carbonitrile as a brownish solid (10.0 g, 82% yield over two steps, 99% pure by LC-MS and ¹H NMR), ¹H NMR (400 MHz; CDCl₃): δ 7.70 (s, 1H); 7.19 (m, 2H); 6.72 (m, 2H); 3.73 (s, 3H); 1.71 (s, 3H).

5-(4-Hydroxy-phenyl)-6-methyl-2-oxo-1,2-dihydropyridine-3-carbonitrile: To a stirred solution of 5-(4-Methoxy-phenyl)-6-methyl-2-oxo-1,2-dihydro-pyridine-3-carbonitrile (10.0 g, 41.6 mmol) in dichloromethane (200 mL) was added dropwise at 0° C. a solution of boron tribromide (11.8 mL, 125 mmol) in DCM (125 mL). The reaction mixture was stirred for 6 hours at ambient temperature, poured into a mixture of ice and saturated ammonium chloride solution (100 mL), and stirred for 1 hour at room temperature. The formed precipitate was filtered off, rinsed with water and re-dissolved in aqueous sodium hydroxide (2 N, 400 mL). The aqueous solution was washed with ethyl acetate (100 mL), acidified to pH 4 with aqueous hydrochloric acid (2 N), and extracted with ethyl acetate (3×200 mL). The combined organic phases were washed with brine (2×200 mL), dried (MgSO₄) and evaporated to dryness to give 5-(4-hydroxy-phenyl)-6-methyl-2-oxo-1,2-dihydro-pyridine-3-carbonitrile as a yellow solid (3.25 g, 46% yield, 92% pure by LC-MS and ¹H NMR), ¹H NMR (400 MHz; CDCl₃): δ 7.70 (s, 1H); 7.13 (m, 2H); 6.68 (m, 2H); 1.71 (s, 3H).

[4-(5-Cyano-2-methyl-6-oxo-1,6-dihydro-pyridin-3-yl)phenoxy]-acetic acid ethyl ester: To a stirred suspension of sodium hydride (60% dispersion in mineral oil, 1.16 g, 29.0 mmol) in N,N-dimethylformamide (50 mL), was added at 0° C. a solution of 5-(4-hydroxy-phenyl)-6-methyl-2-oxo-1,2-dihydro-pyridine-3-carbonitrile (3.25 g, 14.4 mmol) in N,N-dimethylformamide (50 mL). The mixture was stirred at ambient temperature for 30 minutes. A solution of ethyl 2-bromoacetate (2.0 mL, 18.0 mmol) in N,N-dimethylformamide (10 mL) was added at 0° C., the mixture was stirred for 30 minutes at 0° C., for 30 minutes at ambient temperature, and then for 45 minutes at 80° C. The mixture was allowed to cool to room temperature, concentrated in vacuo and re-dissolved in ethyl acetate (300 mL). The solution was extracted with water (3×150 mL). The combined aqueous layers were acidified to pH 2 with aqueous hydrochloric acid (1 N) and extracted with ethyl acetate (3×150 mL). The combined organic layers were dried (MgSO₄) and evaporated to dryness. The residue was purified by column chromatography on silica gel (50 g) using 2% methanol in dichloromethane as eluent to give [4-(5-Cyano-2-methyl-6-oxo-1,6-dihydro-pyridin-3-yl)phenoxy]-acetic acid ethyl ester as light yellow powder (1.3 g, 29% yield, 80-90% pure by LC-MS and ¹H NMR), ¹H NMR (400 MHz; CDCl₃): δ 7.70 (d, 1H); 7.19 (m, 2H); 6.72 (m, 2H); 4.90 (s, 2H); 4.12 (q, 2H); 1.71 (s, 3H); 1.30 (t, 3H).

[4-(5-Cyano-2-methyl-6-oxo-1,6-dihydro-pyridin-3-yl)-phenoxy]-acetic acid: To a stirred solution of [4-(5-Cyano-2-methyl-6-oxo-1,6-dihydro-pyridin-3-yl)phenoxy]-acetic acid ethyl ester (1.3 g, 4.16 mmol) in a mixture of 1,4-dioxane (25 mL) and water (25 mL) was added lithium hydroxide mono hydrate (700 mg, 16.7 mmol). The reaction mixture was stirred for 2 hours at ambient temperature, diluted with water (50 mL), washed with diethylether (2×25 mL), cooled to 0° C. and acidified to pH 2 with aqueous hydrochloric acid (5 N). After standing at ambient temperature overnight the formed precipitate was filtered off with suction, washed with water and dried under vacuum to give [4-(5-cyano-2-methyl-6-oxo-1,6-dihydro-pyridin-3-yl)-phenoxy]-acetic acid as a light yellow crystalline solid (758 mg, 64% yield, 97% pure by LC-MS and ¹H NMR), ¹H NMR (400 MHz; CDCl₃): δ 7.70 (d, 1H); 7.20 (m, 2H); 6.73 (m, 2H); 4.88 (s, 2H); 1.71 (s, 3H).

### Example 7

N-{3-[4-(2-Hydroxy-3-isopropylaminopropoxy)phenoxy]-propyl}-4-(2-oxo-1,2-dihydro-quinolin-6-yloxy)butyramide was synthesized using the same procedure as was used for Example 5, starting from 4-(2-oxo-1,2-dihydro-quinolin-6-yloxy)-butyric acid (110 mg, 0.446 mmol). N-{3-[4-(2-Hydroxy-3-isopropylamino-propoxy)-phenoxy]-propyl}-4-(2-oxo-1,2-dihydro-quinolin-6-yloxy)-butyramide was isolated as an off-white solid (103 mg, 45% yield, 97% pure by LC-MS and ¹H-NMR). ¹H NMR (400 MHz; CDCl₃): δ 7.48 (m, 1H); 7.36 (d, 1H); 6.79 (m, 1H); 6.66 (m, 4H); 6.63 (m, 1H); 6.57 (d, 1H); 4.09 (s, 2H); 3.96 (m, 1H); 3.94 (m, 4H total); 3.20 (m, 2H); 2.97 (m, 1H); 2.70 (m, 2H); 2.18 (m, 2H); 1.99 (m, 2H); 1.97 (m, 2H); 1.05 (d, 6H total).

The required PDE3 inhibitor fragment, 4-(2-oxo-1,2-dihydro-quinolin-6-yloxy)-butyric acid, was synthesized as described in Scheme V-c.

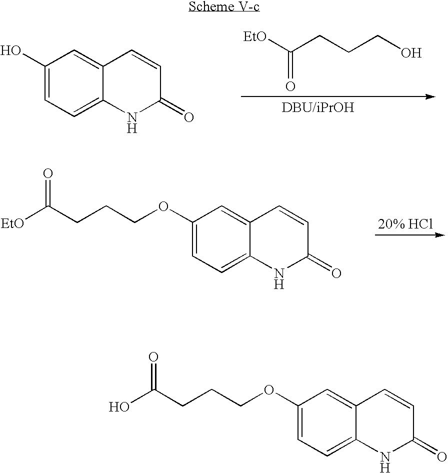

Methyl 4-(2-oxo-6-hydroquinolyloxy)butanoate: Methyl 4-bromobutyrate (6.8 g) was added drop-wise with stirring to a solution of 5 g of 6-hydroxyhydroqionoline-2-one and 7 g of 1,8-diazabicyclo[5.4.0]undec-7-ene (DBU) in 75 mL of isopropanol, and refluxed for 4 hours. After cooling and removal of the solvent under vacuum, the residue was dissolved in methylene chloride and the organic phase was washed successively with 0.5 N NaOH, diluted HCl and water, dried over MgSO₄, and concentrated. Recrystallization of the crude product from water furnished the substituted quinolone as colorless needles, ¹H NMR (400 MHz; CDCl₃): δ 7.48 (m, 1H); 7.36 (d, 1H); 6.79 (m, 1H); 6.63 (m, 1H); 6.57 (d, 1H); 3.94 (m, 2H); 3.67 (s, 3H); 2.25 (m, 2H); 2.10 (m, 2H).

4-(2-oxo-6-hydroquinolyl)butyric acid: A suspension of the methyl ester in 20% HCl was stirred for 2 hours at 90° C., cooled, and the crystals were collected by filtration, washed with cold water, and dried to deliver the acid as a granular solid, ¹H NMR (400 MHz; CDCl₃): δ 7.48 (m, 1H); 7.36 (d, 1H); 6.79 (m, 1H); 6.63 (m, 1H); 6.57 (d, 1H); 3.94 (m, 2H); 2.23 (m, 2H); 1.98 (m, 2H).

### Example 8

### 6-(3-Chloro-4-{3-[4-(2-hydroxy-3-isopropylamino-propoxy)-phenoxy]-propoxy}-phenyl)-4,5-dihydro-2H-pyridazin-3-one was Synthesized According to Scheme VI

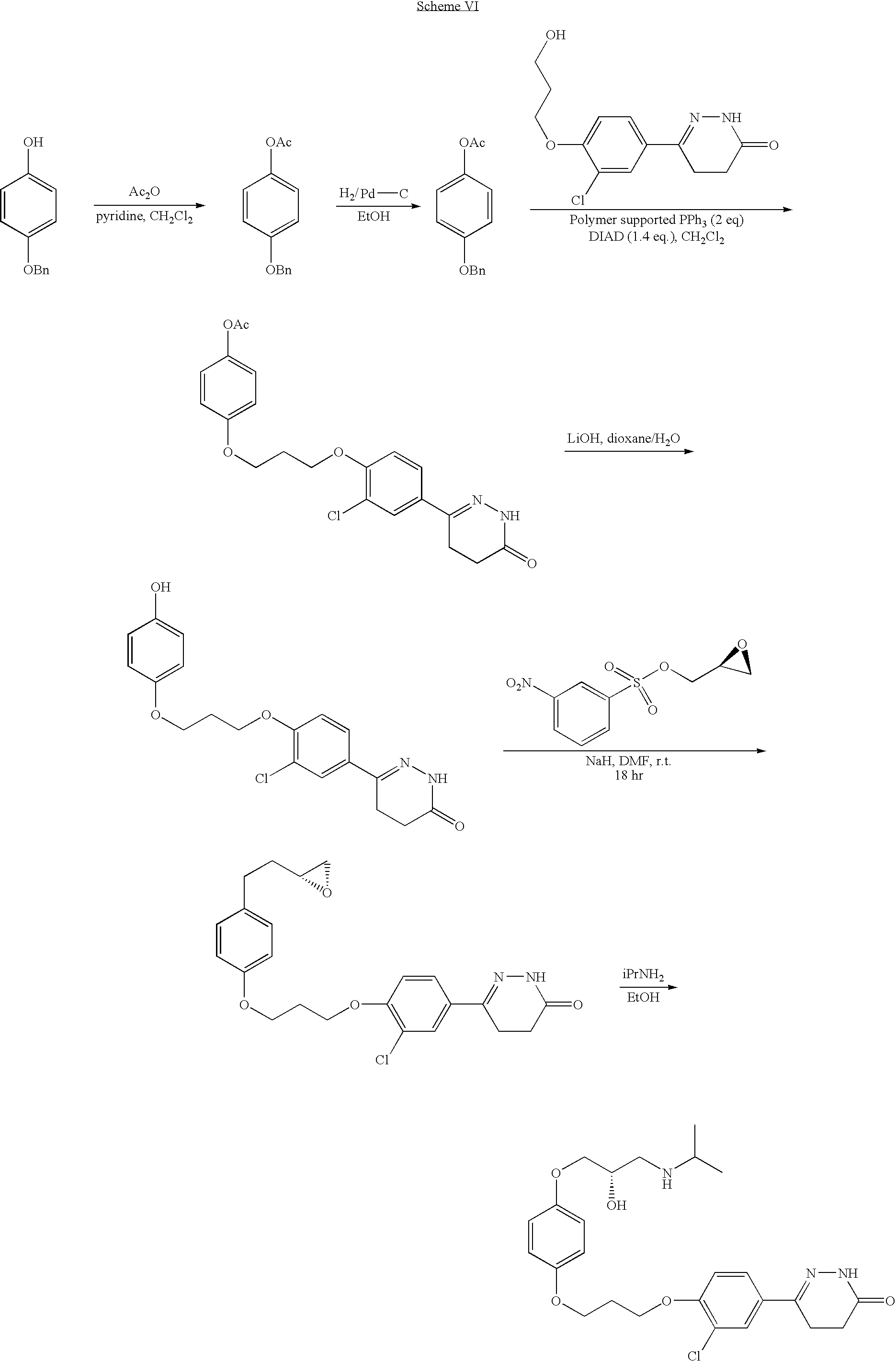

Acetic acid 4-hydroxy-phenyl ester: To a stirred solution of 4-benzyloxy-phenol (4.0 g, 20.0 mmol) in tetrahydrofuran (50 mL) was added pyridine (1.94 ml, 24.0 mmol) and acetic anhydride (2.26 mL, 24.0 mmol). The reaction mixture was heated to reflux and stirred at this temperature for 2 hours, cooled to ambient temperature then poured into ethyl acetate (200 mL). The resultant solution was washed with aqueous hydrochloric acid (0.5 N, 2×50 mL), aqueous sodium carbonate solution (2 N, 2×50 mL) and saturated brine (2×50 mL). The organic layer was dried (Na₂SO₄) and concentrated under reduced pressure to give crude acetic acid 4-benzyloxy-phenyl ester. This product was dissolved in ethanol/tetrahydrofuran (5:1) (300 mL) under nitrogen and to the solution was added palladium on carbon (10 wt % palladium, 50% wet Degussa type, 1.80 g, 0.85 imnol). The reaction mixture was stirred at ambient temperature for 2 hours under hydrogen atmosphere (1.5 atm) and then filtered through Celite®. The filtrate was concentrated under reduced pressure to give acetic acid 4-hydroxy-phenyl ester as a pale yellow oil (2.76 g, 91% yield, 99% pure by LC-MS and ¹H-NMR, no mass ion found). ¹H NMR (300 MHz, CDCl₃): δ 6.90 (d, 2H); 6.70 (d, 2H); 2.08 (s, 3H).

Acetic acid 4-{3-[2-chloro-4-(6-oxo-1,4,5,6-tetrahydro-pyridazin-3-yl)-phenoxy]-propoxy}phenyl ester: To a stirred suspension of acetic acid 4-hydroxy-phenyl ester (211 mg, 1.39 mmol) in dry dichloromethane under nitrogen was added 6-[3-chloro-4-(3-hydroxy-propoxy)-phenyl]-4,5-dihydro-2H-pyridazin-3-one (302 mg, 1.07 mmol) and triphenylphosphine resin (polystyrene bound, 1.20 mmol/g loading, 1.80 g 2.16 mmol). The mixture was stirred at −10° C. for 10 minutes, then diisopropyl azodicarboxylate (DIAD, 310 μL, 1.57 mmol) was added and the reaction mixture was allowed to warm to ambient temperature with stirring, then stirred at this temperature for 16 hours. The mixture was filtered and the filtered residue rinsed alternately with dichloromethane (5 mL) and methanol (5 mL) (×3). The combined filtrates were evaporated to dryness and the residue was dry-loaded and purified by column chromatography on silica gel (20 g), eluting with a gradient of hexane/ethyl acetate (1: 1) to neat ethyl acetate. Fractions with R_{f}=0.46 (EtOAc) were combined and concentrated under reduced pressure to give acetic acid 4-{3-[2-chloro-4-(6-oxo-1,4,5,6-tetrahydropyridazin-3-yl)-phenoxy]-propoxy}-phenyl ester as a colorless oil (393 mg, 88% yield, 90% pure by LC-MS and ¹H-NMR). ¹H NMR (300 MHz, CDCl₃): δ 7.51 (d, 1H); 7.42 (dd, 1H); 6.96 (dd, 2H); 6.69 (dd, 1H); 6.74 (dd, 2H); 3.94 (broad m, 4H total); 2.21 (m, 2H); 2.13 (m, 2H); 2.08 (s, 3H); 1.61 (m, 2H).

6-{3-Chloro-4-[3-(4-hydroxy-phenoxy)-propoxyl]-phenyl}-4,5-dihydro-2H-pyridazin-3-one: To a stirred solution of acetic acid 4-{3-[2-chloro-4-(6-oxo-1,4,5,6-tetrahydro-pyridazin-3-yl)-phenoxy]-propoxy}-phenyl ester (393 mg, 0.94 mol) in tetrahydrofuran (5 mL), H₂O (4 mL) and methanol (1 mL) was added lithium hydroxide monohydrate (80.0 mg, 1.91 mmol). The reaction mixture was stirred at ambient temperature under nitrogen atmosphere for 18 hours, quenched with glacial acetic acid (0.5 mL), and adsorbed onto silica gel (2 g). The mixture was evaporated to dryness under reduced pressure and dry-loaded onto a silica gel column (10 g). Purification by column chromatography was carried out using hexane/ethyl acetate (20:80) as eluent. Fractions with R_{f}=0.40 (EtOAc) were combined and evaporated to dryness. The residue was triturated with chloroform (1 mL) and dried under reduced pressure to give 6-{3-chloro-4-[3-(4-hydroxy-phenoxy)-propoxy)-phenyl}-4,5-dihydro-2H-pyridazin-3-one as a colorless solid (230 mg, 65% yield, 99 pure by LC-MS and ¹H-NMR). ¹H NMR (300 MHz, CDCl₃): δ 7.50 (d, 1H); 7.41 (dd, 1H); 6.70 (dd, 1H); 6.62 (dd, 2H); 6.60 (dd, 2H); 3.94 (m, 4H total); 2.22 (m, 2H); 2.13 (m, 2H); 1.62 (m, 2H).

6-{3-Chloro-4-[3-(4-oxiranylmethoxy-phenoxy)-propoxyl]-phenyl}-4,5-dihydro-2H-pyridazin-3-one: To a stirred suspension of sodium hydride (60% dispersion in mineral oil, 23.0 mg, 0.58 mmol) in N,N-dimethylformamide (5 mL) under nitrogen at 0° C. was added 6-{3-chloro-4-[3-(4-hydroxy-phenoxy)-propoxy]-phenyl}-4,5-dihydro-2H-pyridazin-3-one (215 mg, 0.57 mmol) and the reaction mixture was stirred for 20 minutes at ambient temperature. A solution of 3-nitro-benzenesulfonic acid oxiranylmethyl ester (150 mg, 0.58 mmol) in N,N-dimethylformamide (2 mL) was added at 0° C. The mixture was stirred at ambient temperature for 16 hours, poured onto a mixture of ice and saturated aqueous ammonium chloride solution (25 mL), and extracted with ethyl acetate (3×20 mL). The combined organic layers were washed with saturated brine (3×10 mL), dried Na₂SO₄) and concentrated under reduced pressure to give crude 6-{3-chloro-4-[3-(4-oxiranylmethoxy-phenoxy)-propoxy]-phenyl}4,5-dihydro-2H-pyridazin-3-one as a yellow gum, which was used without further purification in the next step.

6-(3-Chloro-4-{3-[4-(2-hydroxy-3-isopropylamino-propoxy)-phenoxy]-propoxy}-phenyl)-4,5-dihydro-2H-pyridazin-3-one: To a stirred suspension of crude 6-{3-chloro-4-[3-(4-oxiranylmethoxy-phenoxy)-propoxy]-phenyl}-4,5-dihydro-2H-pyridazin-3-one in ethanol (5 mL) was added iso-propylamine (490 μL, 5.74 mmol). The reaction mixture was heated to reflux and stirred at this temperature for 2 hours, allowed to cool to ambient temperature and evaporated to dryness under reduced pressure. The residue was dry-loaded and purified by column chromatography on silica gel (3 g) using a gradient of dichloromethane/methanol (9:1) to dichloromethane/methanol (4: 1) as eluent. Fractions with R_{f}=0.05 were combined and concentrated under reduced pressure. The residue was recrystallised from ethanol to give 6-(3-chloro-4-{3-[4-(2-hydroxy-3-isopropylaminopropoxy)phenoxy]propoxy}-phenyl)-4,5-dihydro-2H-pyridazin-3-one (Example 8) as an off white solid (128 mg, 46% yield over two steps, 98% pure by LC-MS and ¹H-NMR). ¹H NMR (300 MHz, CDCl₃): δ 7.51 (d, 1H); 7.40 (d, 1H); 6.71 (d, 1H); 6.66 (m, 4H); 4.09 (d, 2H); 3.96 (m, 1H); 3.94 (m, 4H); 2.97 (q, 1H); 2.70 (m, 2H); 2.21 (m, 2H); 2.13 (m, 2H); 1.61 (m, 2H); 1.05 (d, 6H total).

The required pyridazinone glycol was prepared according to the method of Scheme VI-a.

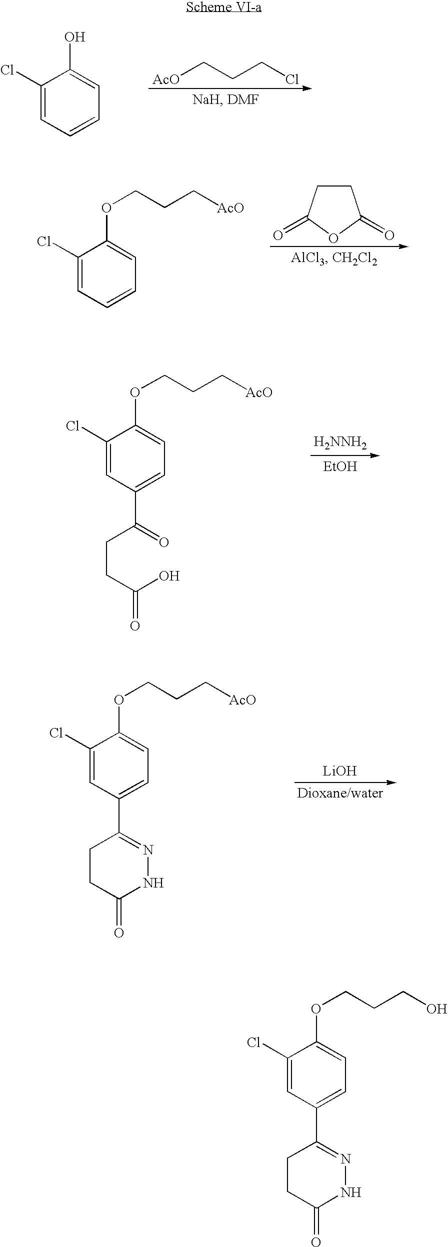

Acetic acid 3-(2-chloro-phenoxy)-propyl ester: To a stirred suspension of sodium hydride (60% dispersion in mineral oil, 7.40 g, 185 mmol) in N,N-dimethylformamide (150 mL) under nitrogen was added portionwise a solution of 2-chlorophenol (16.0 mL, 154 mmol) in N,N-dimethylformamide (50 mL) at 0° C. The reaction mixture was stirred for 30 minutes at ambient temperature and a solution of acetic acid 3-chloro-propyl ester (21.0 mL, 170 mmol) in N,N-dimethylformamide (50 mL) was added. The reaction mixture was stirred for 30 minutes at ambient temperature and then for 16 hours at 50° C. After cooling to ambient temperature, the reaction mixture was poured into a mixture of ice and saturated aqueous ammonium chloride solution (250 mL), and extracted with ethyl acetate (4×100 mL). The combined organic layers were washed with aqueous sodium hydroxide solution (1 N, 100 mL) and brine (2×100 mL), dried (MgSO₄) and evaporated to dryness to give acetic acid 3-(2-chloro-phenoxy)-propyl ester as a light orange oil (31.8 g, 90% yield, 93% pure by LC-MS and ¹H-NMR). ¹H NMR (400 MHz, CDCl₃): δ 7.16 (m, 1H); 7.03 (m, 1H); 6.75-6.71 (m, 2H); 4.08 (m, 2H); 3.94 (m, 2H); 2.01 (s, 3H); 1.99 )m, 2H).

4-[4-(3-Acetoxy-propoxy)-3-chloro-phenyl]-4-oxo-butyric acid: To a stirred solution of acetic acid 3-(2-chloro-phenoxy)-propyl ester (31.8 g, 139 mmol) in dichloromethane (100 mL) at ambient temperature under nitrogen was added succinic anhydride (20.8 g, 208 mmol). The reaction mixture was cooled in ice-water and aluminum trichloride (55.6 g, 417 mmol) was added portionwise whilst maintaining the temperature below 20° C. The yellow suspension was stirred at ambient temperature for 20 minutes and then at 50° C. for 16 hours. The obtained dark purple highly viscous oil was allowed to cool to ambient temperature and then carefully hydrolysed with ice-water (100 ml) and ice-aqueous hydrochloric acid (10 N, 100 ml). The aqueous layer was extracted with ethyl acetate (5×100 mL). The combined organic layers were washed with saturated brine (2×100 mL), dried (Na₂SO₄), and concentrated under reduced pressure to give an orange oil. The residue was re-dissolved in hot ethyl acetate (50 mL), hexane (200 mL) was added and the mixture was shaken for 10 minutes. After standing at ambient temperature for 1 hour, the supernatant was decanted. The residue was rinsed with 100 mL hexane and dried under reduced pressure at 50° C. to give 4-[4-(3-acetoxy-propoxy)-3-chloro-phenyl]-4-oxo-butyric acid as a yellow gum (42.7 g, 93% yield, 90% pure by LC-MS and ¹H-NMR). ¹H NMR (400 MHz, CDCl₃): δ 7.79 (m, 1H); 7.66 (m, 1H); 6.79 (m, 1H); 4.08 (m, 2H); 3.94 (m, 2H); 2.82 (m, 2H); 2.42 (m, 2H); 2.01 (s, 3H); 1.99 (m, 2H).

Acetic acid 3-[2-chloro-4-(6-oxo-1,4,5,6-tetrahydro-pyridazin-3-yl)-phenoxy]-propyl ester: To a stirred suspension of 4-[4-(3-acetoxy-propoxy)-3-chloro-phenyl]-4-oxo-butyric acid (42.7 g, 130 mmol) in ethanol (300 mL) at 0° C. was added a solution of hydrazine monohydrate (5.74 mL, 117 mmol) in ethanol (50 mL). The reaction mixture was allowed to warm to ambient temperature and stirred at this temperature for 15 minutes before being heated to reflux and stirred at this temperature for 3 hours. Ethyl acetate (60 mL) was added to the hot solution and the mixture was allowed to cool to ambient temperature. The precipitate which formed was filtered off and washed with water (2×100 mL) and cold ethanol (2×100 mL), then dried with suction, and then under high vacuum to give acetic acid 3-[2-chloro-4-(6-oxo-1,4,5,6-tetrahydro-pyridazin-3-yl)-phenoxy]-propyl ester as light yellow powder (24.5 g, 58% yield, 97% pure by LC-MS and ¹H-NMR). ¹H NMR (400 MHz, CDCl₃): δ 7.52 (m, 1H); 7.40 (m, 1H); 6.72 (m, 1H); 4.08 (m, 2H); 3.94 (m, 2H); 2.22 (d, 1H); 2.01 (s, 3H); 1.99 (m, 2H); 1.63 (m, 2H).

6-[3-Chloro-4-(3-hydroxy-propoxy)-phenyl]-4,5-dihydro-2H-pyridazin-3-one: To a stirred suspension of acetic acid 3-[2-chloro-4-(6-oxo-1,4,5,6-tetrahydro-pyridazin-3-yl)-phenoxy]-propyl ester (24.5 g, 75.4 mmol) in 1,4-dioxane (125 mL) at ambient temperature were added water (125 mL) and lithium hydroxide (12.7 g, 302 mmol). The reaction mixture was stirred at ambient temperature for 3 hours and then acidified to pH 1-2 with aqueous hydrochloric acid (5 N, 100 mL) with stirring. After standing at ambient temperature for 1 hour, the precipitate was filtered off and washed with water (2×100 mL) and cold ethanol (2×100 mL). The solid was dried under reduced pressure at 45° C. to give 6-[3-chloro-4-(3-hydroxy-propoxy)-phenyl]-4,5-dihydro-2H-pyridazin-3-one as off-white powder (19.2 g, 90% yield, 99% pure by LC-MS and ¹H-NMR). ¹H-NMR (400 MHz, CDCl₃): δ 7.52 (m, 1H); 7.40 (m, 1H); 6.72 (m, 1H); 3.94 (m, 2H); 3.53 (m, 2H); 2.21 (d, 2H); 1.90 (m, 2H); 1.60 (m, 2H)

### Example 9

### N-[3-(4-{(2S)-2-hydroxy-3-[(methylethyl)amino]propoxy}-3-bromophenoxy)propyl]-2-[2-chloro-4-(6-oxo(1,4,5-trihydropyridazin-3-yl))phenoxy]acetamide was Prepared According to the Method of Scheme VII

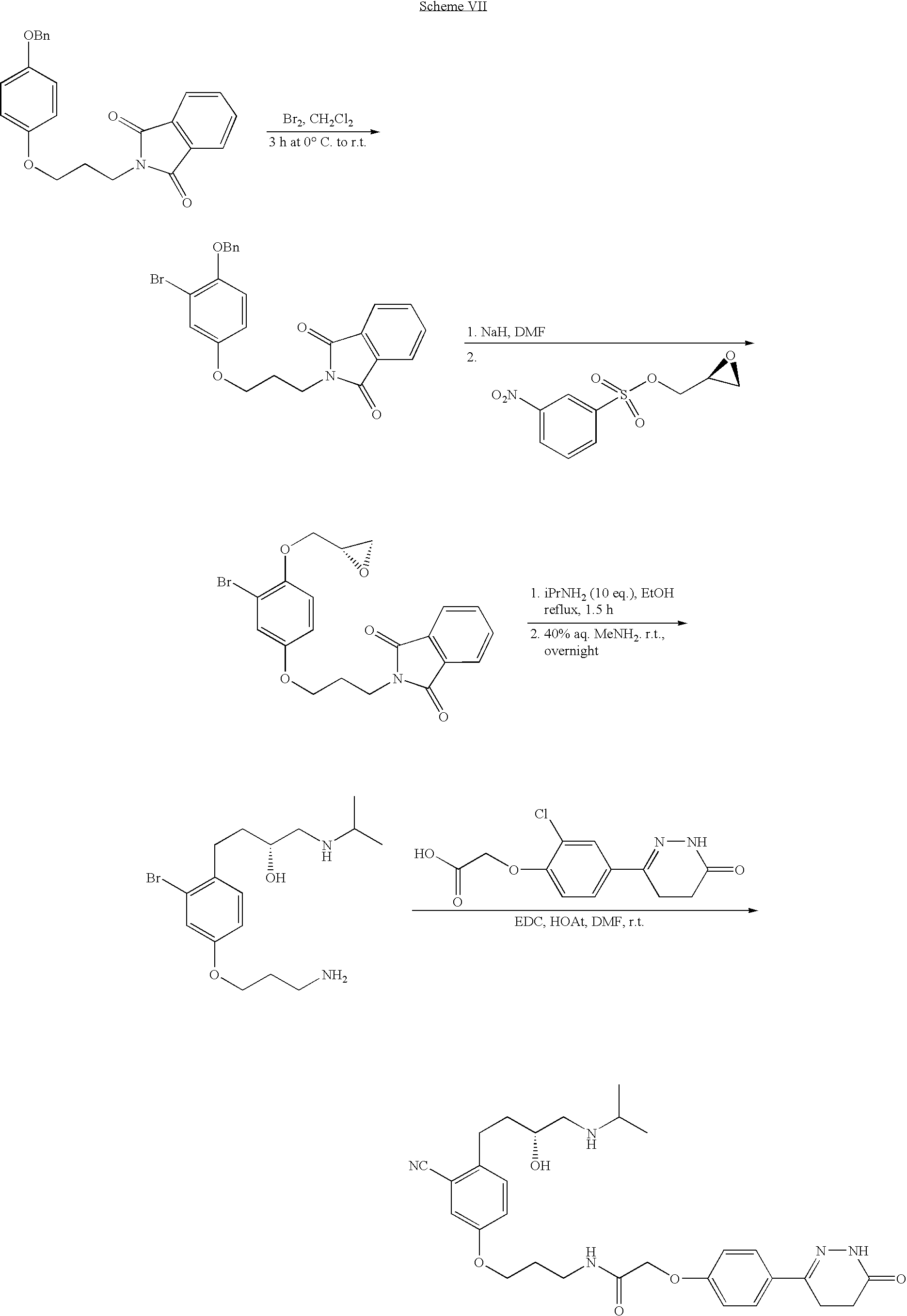

2-[3-(3-Bromo-4-hydroxy-phenoxy)-propyl]-isoindole-1,3-dione: To a stirred solution of 2-[3-(4-hydroxy-phenoxy)-propyl]-isoindole-1,3-dione (1.20 g, 4.04 mmol) in dichloromethane (100 mL) was added dropwise a solution of bromine (210 μL, 4.04 mmol) in dichloromethane (30 mL) at 0-5° C. The reaction mixture was stirred at 5° C. for 3 hours. The precipitate which formed was filtered off, rinsed with cold dichloromethane (10 mL) and dried under reduced pressure to give 2-[3-(3-bromo-4-hydroxy-phenoxy)-propyl]-isoindole-1,3-dione as a colorless solid (870 mg, 57% yield, 98% pure by LC-MS and ¹H-NMR). The filtrate was washed with aqueous sodium sulfite solution (5 wt %, 20 mL) and water (2×50 mL), dried (MgSO₄) and concentrated under reduced pressure to give a second batch of 2-[3-(3-bromo-4-hydroxy-phenoxy)-propyl]-isoindole-1,3-dione as a light yellow powder (560 mg, 36% yield, 90% pure by LC-MS and ¹H-NMR).

2-[3-(3-Bromo-4-(S)-oxiranylmethoxy-phenoxy)-propyl]-isoindole-1,3-dione: To a stirred suspension of sodium hydride (60% dispersion in mineral oil, 35 mg, 0.877 mmol) in N,N-dimethylformamide (4 mL) under nitrogen at 0° C. was added a solution of 2-[3-(3-bromo-4-hydroxy-phenoxy)-propyl]-isoindole-1,3-dione (300 mg, 0.797 mmol) in N,N-dimethylformamide (2 mL) and the reaction mixture was stirred at ambient temperature for 20 minutes. A solution of (2S)-glycidyl m-nitrobenzenesulfonate (207 mg, 0.797 mmol) in N,N-dimethylformamide (2 mL) was added at 0° C. The mixture was stirred at ambient temperature for 16 hours, poured onto a mixture of ice and saturated aqueous ammonium chloride solution (20 mL) and extracted with ethyl acetate (5×30 mL). The combined organic layers were washed with saturated brine (2×30 mL), dried (Na₂SO₄) and concentrated under reduced pressure to give crude 2-[3-(3-bromo-4-(S)-oxiranylmethoxy-phenoxy)-propyl]-isoindole-1,3-dione as a yellow gum, which was used without further purification in the next step.

1-[4-(3-Amino-propoxy)-2-bromo-phenoxy]-3-isopropylamino-(S)-propan-2-ol: To a stirred solution of crude 2-[3-(3-bromo-4-(S)-oxiranylmethoxy-phenoxy)-propyl]-iso-indole-1,3-dione from the previous step in ethanol (10 mL) was added iso-propylamine (700 μL, 8.22 mmol). The reaction mixture was heated to reflux and stirred at this temperature for 3 hours, allowed to cool to ambient temperature then concentrated under reduced pressure. The residue was dissolved in methylamine (40 wt % in water, 10 mL), stirred at 30° C. for 16 hours, diluted with water (20 mL) and saturated brine (20 mL) and extracted with dichloromethane (3×20 mL). The combined organic layers were washed with saturated brine (2×10 mL), dried (Na₂SO₄) and concentrated under reduced pressure to give crude 1-[4-(3-amino-propoxy)-2-bromo-phenoxy]-3-isopropylamino-(S)-propan-2-ol as a colorless oil (230 mg, 80% yield over three steps, 90% pure by LC-MS and ¹H-NMR), which solidified on standing.

N-{3-[3-Bromo-4-((2S)-hydroxy-3-isopropylamino-propoxy)-phenoxy]-propyl}-2-[2-chloro-4-(6-oxo-1,4,56-tetrahydro-pyridazin-3-yl)-phenoxy]-acetamide: To a stirred solution of [2-chloro-4-(6-oxo-1,4,5,6-tetrahydro-pyridazin-3-yl)-phenoxy]-acetic acid (162 mg, 0.573 mmol), 1-(3-dimethylaminopropyl)-3-ethyl carbodiimide hydrochloride (EDC.HCl, 110 mg, 0.573 mmol) and 7-hydroxyazabenzotriazole (HOAt, 78 mg, 0.573 mmol) in N,N-dimethylformamide (2.5 mL) under N₂ was added a solution of 1-[4-(3-amino-propoxy)-2-bromo-phenoxy]-3-isopropylamino-(S)-propan-2-ol (230 mg, 0.637 mmol) in N,N-dimethylformamide (2.5 mL). The reaction mixture was stirred at ambient temperature for 3 hours, poured into saturated brine (20 mL), made strongly alkaline (pH 11-12) with aqueous sodium hydroxide solution (2 N), and extracted with ethyl acetate (5×20 mL). The combined organic layers were washed with saturated brine (2×10 mL), dried (Na₂SO₄) and concentrated under reduced pressure. The residue was purified by flash column chromatography over silica gel (3 g) eluting with dichloromethane/methanol (9:1). Fractions with R_{f}=0.09 were combined and concentrated under reduced pressure to give N-{3-[3-bromo-4-((28)-hydroxy-3-isopropylamino-propoxy)-phenoxy]-propyl}-2-[2-chloro-4-(6-oxo-1,4,5,6-tetrahydro-pyridazin-3-yl)-phenoxy]-acetamide as a colorless powder (130 mg, 33% yield, 95% pure by LC-MS and ¹H-NMR).

### Example 10

### N-[3-(4-{(2S)-2-hydroxy-3-[(methylethyl)amino]propoxy}-3-cyanophenoxy)propyl]-2-[2-chloro-4-(6-oxo(1,4,5-trihydropyridazin-3-yl))phenoxy]acetamide was Prepared According to Scheme VIII

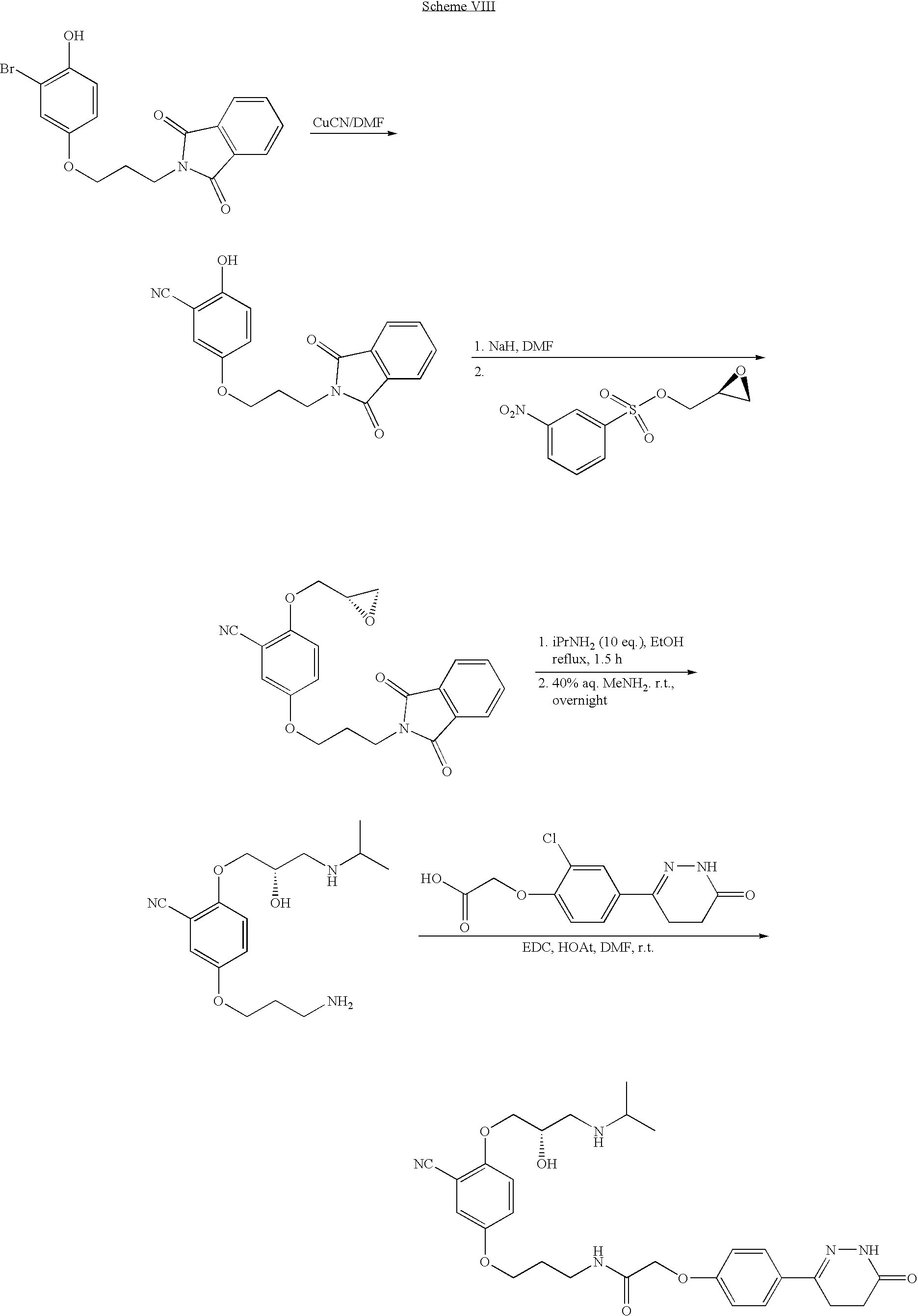

5-[3-(1 3-Dioxo-1,3-dihydro-isoindol-2-yl)-propoxy]-2-hydroxy-benzonitrile: To a stirred solution of 2-[3-(3-bromo-4-hydroxy-phenoxy)-propyl]-isoindole-1,3-dione (550 mg, 1.46 mmol) in N,N-dimethylformamide (10 mL) was added copper (I) cyanide (160 mg, 1.75 mmol). The reaction mixture was then heated to 155° C. under nitrogen and stirred at this temperature for 9 hours. After allowing to cool to ambient temperature the solution was diluted with ethyl acetate (20 mL). A solution of ethylenediaminetetraacetic acid (850 mg, 2.91 mmol) in water (20 mL) was added and the resulting suspension was stirred at ambient temperature for 1 hour. The two phases were separated and the aqueous layer was extracted with ethyl acetate (3×20 mL). The combined organic layers were washed with water (3×20 mL), dried (MgSO₄) and concentrated under reduced pressure. The residue was taken and filtered through a pad of silica gel (2 g) eluting with ethyl acetate. The filtrate was evaporated to dryness under reduced pressure to give 5-[3-(1,3-dioxo-1,3-dihydro-isoindol-2-yl)-propoxy]-2-hydroxy-benzonitrile as a brown powder (330 mg, 70% yield, 85% pure by LC-MS and ¹H-NMR).

5-[3-(1,3-Dioxo-1,3-dihydro-isoindol-2-yl)-propoxy]-2-(S)-oxiranylmethoxy-benzonitrile: To a stirred suspension of sodium hydride (60% dispersion in mineral oil, 33 mg, 0.819 mmol) in N,N-dimethylformamide (2 mL) under nitrogen at 0° C. was added a solution of 5-[3-(1,3-dioxo-1,3-dihydro-isoindol-2-yl)-propoxy]-2-hydroxy-benzonitrile (240 mg, 0.745 mmol) in N,N-dimethylformamide (2 mL) and the reaction mixture was stirred at ambient temperature for 10 minutes. A solution of (2S)-glycidyl m-nitrobenzenesulfonate (193 mg, 0.745 mmol) in N,N-dimethylformamide (2 mL) was added at 0° C. The reaction mixture was stirred at ambient temperature for 4 hours, poured onto a mixture of ice-water (10 mL) and saturated aqueous ammonium chloride solution (10 mL) and extracted with ethyl acetate (3×20 mL). The combined organic layers were washed with a mixture of saturated brine (10 mL) and saturated aqueous sodium hydrogen carbonate solution (10 mL) and then with saturated brine (2×20 mL). The organic layer was dried (Na₂SO₄) and concentrated under reduced pressure to give crude 5-[3-(1,3-dioxo-1,3-dihydro-isoindol-2-yl)-propoxy]-2-(S)-oxiranylmethoxy-benzonitrile (255 mg) as a light yellow solid, which was used in the next step without further purification.

5-(3-Amino-propoxy)-2-((2S)-hydroxy-3-isopropylamino-propoxy)-benzonitrile: To a stirred solution of crude 5-[3-(1,3-dioxo-1,3-dihydro-isoindol-2-yl)-propoxy]-2-(S)-oxiranylmethoxy-benzonitrile in ethanol (10 mL) was added iso-propylamine (560 μL, 6.74 mmol). The reaction mixture was heated to reflux and stirred at this temperature for 3 hours then concentrated under reduced pressure. The residue was dissolved in methylamine (40 wt % in water, 10 mL) and the resulting solution was heated to 30° C. and stirred at this temperature for 16 hours. After cooling to ambient temperature the solution was diluted with water (20 mL) and saturated brine (20 mL) and extracted with dichloromethane (3×20 mL). The combined organic extracts were washed with saturated brine (2×10 mL), dried (Na₂SO₄) and concentrated under reduced pressure to give 5-(3-amino-propoxy)-2-((2S)-hydroxy-3-isopropylamino-propoxy)-benzonitrile as a yellow oil (140 mg, 67% yield over three steps, 90% pure by LC-MS and ¹H-NMR), which solidified on standing.

2-[2-Chloro-4-(6-oxo-1,4,5,6-tetrahydro-pyridazin-3-yl)-phenoxy]-N-{3-[3-cyano-4-((2S)-hydroxy-3-isopropylamino-propoxy)-phenoxy]-propyl}-acetamide hydrochloride: To a stirred solution of [2-chloro-4-(6-oxo-1,4,5,6-tetrahydro-pyridazin-3-yl)-phenoxy]-acetic acid (116 mg, 0.410 mmol), 1-(3-dimethylaminopropyl)-3-ethyl carbodiimide hydrochloride (EDC.HCl, 78 mg, 0.410 mmol) and 7-hydroxyazabenzotriazole (HOAt, 56 mg, 0.410 mmol) in N,N-dimethylformamide (2.5 mL) under nitrogen was added a solution of 5-(3-amino-propoxy)-2-((2S)-hydroxy-3-isopropylamino-propoxy)-benzonitrile (140 mg, 0.455 mmol) in N,N-dimethylformamide (2.5 mL). The reaction mixture was stirred at ambient temperature for 3 hours, diluted with water (10 mL), adjusted to pH 6 with aqueous hydrochloric acid (1 N), and washed with ethyl acetate (2×10 mL). The aqueous layer was left to stand at 5-10° C. for 16 hours. The precipitate which formed was filtered off, washed with water (2×10 mL) and dried under reduced pressure at 50° C. to give 2-[2-chloro-4-(6-oxo-1,4,5,6-tetrahydro-pyridazin-3-yl)-phenoxy]-N-{3-[3-cyano-4-((2S)-hydroxy-3-isopropylamino-propoxy)-phenoxy]-propyl}-acetamide hydrochloride as a colorless powder (80 mg, 34% yield, 99% pure by LC-MS and ¹H-NMR).

### Example 11

### N-[3-(4-{(2S)-2-hydroxy-3-[(methylethyl)amino]propoxy}-2cyanophenoxy)propyl]-2-[2-chloro-4-(6-oxo(1,4,5-trihydropyridazin-3-yl))phenoxy]acetamide was Prepared According to Scheme IX

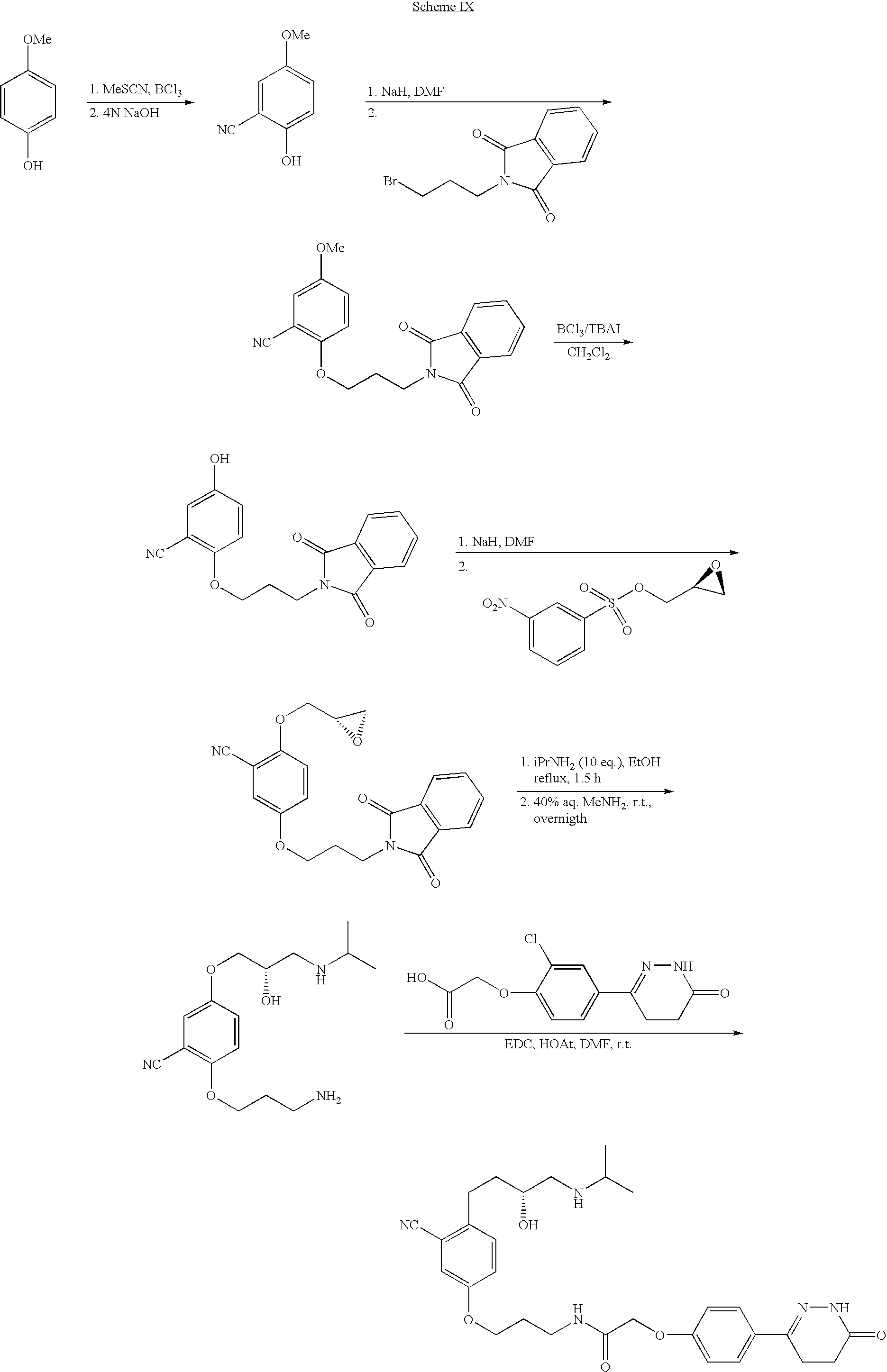

2-Hydroxy-5-methoxybenzonitrile: To a stirred solution of 4-methoxyphenol (12.4 g, 0.10 mol) in dry dichloromethane (400 ml) under nitrogen at 0° C. was added boron trichloride (1 M in dichloromethane, 100 mL, 0.10 mol) followed by methyl thiocyanate (8.2 mL, 0.12 mol). Anhydrous aluminium chloride (2.0 g, 15 mmol) was then added and the resulting suspension was stirred at ambient temperature for 16 hours. The reaction mixture was then cooled to 0° C. and cold aqueous sodium hydroxide solution (4 N, 350 mL) was added. The resulting mixture was then heated to reflux and the dichloromethane was collected by distillation. After cooling to ambient temperature, cold aqueous hydrochloric acid (6 N, 300 mL) was added and the mixture was extracted with diethyl ether (3×200 mL). The combined organic extracts were washed with saturated brine (2×300 mL) and dried (Na₂SO₄) and concentrated under reduced pressure to give a pale yellow solid (15 g) with was purified by flash column chromatography over silica gel to give 2-hydroxy-5-methoxybenzonitrile as a pale yellow solid (10.4 g, 70% yield, 100% pure by LC-MS and ¹H-NMR).

2-[3-(1,3-Dioxo-1,3-dihydro-isoindol-2-yl)-propoxy]-5-methoxy-benzonitrile: To a stirred suspension of sodium hydride (60% dispersion in mineral oil, 450 mg, 11.3 mmol) in N,N-dimethylformamide (10 mL) under nitrogen at 0° C. was added portionwise a solution of 2-hydroxy-5-methoxy-benzonitrile (1.40 g, 9.39 mmol) in N,N-dimethylformamide (10 mL) and the reaction mixture was stirred at ambient temperature for 10 minutes. A solution of 2-(3-bromopropyl)-isoindole-1,3-dione (2.82 g, 10.5 mmol) in N,N-dimethylformamide (20 mL) was added at 0° C. and the reaction mixture was stirred at ambient temperature for 16 hours, poured into ice-water (200 mL) and left to stand at ambient temperature for 15 minutes. The formed precipitate was filtered off with suction, washed with water (25 mL) and diethyl ether (25 mL) then dried under reduced pressure to give 2-[3-(1,3-dioxo-1,3-dihydro-isoindol-2-yl)-propoxy]-5-methoxy-benzonitrile as a light yellow solid (2.51 g, 79% yield, 99% pure by LCMS and ¹H-NMR).

2-[3-(1,3-Dioxo-1,3-dihydro-isoindol-2-yl)-propoxy]-5-hydroxy-benzonitrile: To a stirred solution of 2-[3-(1,3-dioxo-1,3-dihydro-isoindol-2-yl)-propoxy]-5-methoxy-benzonitrile (1.09 g, 3.24 mmol) and tetra-n-butylammonium iodide (1.28 g, 3.47 mmol) in dry dichloromethane (20 ml) at −78° C. was added boron trichloride (1 M in dichloromethane, 14.6 mL, 14.6 mmol) maintaining the internal temperature below −60° C. The reaction mixture was stirred at −78° C. for 10 minutes, allowed to warm to ambient temperature then stirred for a further 2 hours at ambient temperature. The mixture was then poured onto cold saturated aqueous sodium hydrogen carbonate solution (80 mL). The organic layer was separated and the aqueous layer was extracted with dichloromethane (2×50 mL). The combined organic layers were washed with water (100 mL), saturated brine (2×100 mL), dried (Na₂SO₄) and concentrated under reduced pressure. The resulting residue was purified by flash column chromatography over silica gel eluting with dichloromethane/methanol (99.5:0.5) to give 2-[3-(1,3-dioxo-1,3-dihydro-isoindol-2-yl)-propoxy]-5-hydroxy-benzonitrile as a colorless solid (773 mg, 74% yield, 99% pure by LC-MS and ¹H-NMR).

2-[3-(1,3-Dioxo-1,3-dihydro-isoindol-2-yl)-propoxy-5-(S)-oxiranylmethoxy-benzonitrile: To a stirred suspension of sodium hydride (60% dispersion in mineral oil, 49 mg, 1.23 mmol) in N,N-dimethylformamide (2 mL) under nitrogen at 0° C. was added a solution of 2-[3-(1,3-dioxo-1,3-dihydro-isoindol-2-yl)-propoxy]-5-hydroxy-benzonitrile (369 mg, 1.14 mmol) in N,N-dimethylformamide (2 mL) and the reaction mixture was stirred at ambient temperature for 10 minutes. A solution of (2S)-glycidyl m-nitrobenzenesulfonate (7, 323 mg, 1.25 mmol) in N,N-dimethylformamide (2 mL) was then added at 0° C. The reaction mixture was stirred at ambient temperature for 16 hours then poured onto a mixture of ice-water (15 mL) and saturated aqueous ammonium chloride solution (15 mL), and the resulting mixture was extracted with ethyl acetate (4×20 mL). The combined organic extracts were washed with water (2×50 mL) and saturated brine (50 mL), dried (Na₂SO₄) and concentrated under reduced pressure. The residue was purified by flash column chromatography over silica gel using a gradient eluent neat dichloromethane to dichloromethane/ethyl acetate (9:1) to give 2-[3-(1,3-dioxo-1,3-dihydro-isoindol-2-yl)-propoxy]-5-(S)-oxiranylmethoxy-benzonitrile as a colorless solid (362 mg, 84% yield, 99% pure by LC-MS and ¹H-NMR).

2-(3-Amino-propoxy)-5-((2S)-hydroxy-3-isopropylamino-propoxy)-benzonitrile: To a stirred solution of 2-[3-(1,3-dioxo-1,3-dihydro-isoindol-2-yl)-propoxy]-5-(S)-oxiranylmethoxy-benzonitrile (240 mg, 0.634 mmol) in ethanol (7 mL) was added iso-propylamine (540 μL, 6.34 mmol). The reaction mixture was heated to reflux and stirred at this temperature for 2 hours. After allowing to cool to ambient temperature, the solution was then concentrated under reduced pressure. The residue was dissolved in methylamine (40 wt % in water, 7 mL), heated to 30° C. and stirred at this temperature for 16 hours. After cooling to ambient temperature, the solution was diluted with water (10 mL) and saturated brine (10 mL) then extracted with dichloromethane (4×10 mL). The combined organic extracts were washed with water (2×10 mL) and saturated brine (2×20 mL), dried (Na₂SO₄) and concentrated under reduced pressure to give crude 2-(3-amino-propoxy)-5-((2S)-hydroxy-3-isopropylamino-propoxy)-benzonitrile as a colorless oil (176 mg, 90% yield, 90% pure by LC-MS and ¹H-NMR), which solidified on standing.

2-[2-Chloro-4-(6-oxo-1,4,5,6-tetrahydro-pyridazin-3-yl)-phenoxy]-N-{3-[2-cyano-4-((2S)-hydroxy-3-isopropylamino-propoxy)-phenoxy]-propyl}-acetamide hydrochloride: To a stirred solution of [2-chloro-4-(6-oxo-1,4,5,6-tetrahydro-pyridazin-3-yl)-phenoxy]-acetic acid (146 mg, 0.515 mmol), 1-(3-dimethylaminopropyl)-3-ethyl carbodiimide hydrochloride (EDC.HCl, 99 mg, 0.515 mmol) and 7-hydroxyazabenzotriazole (HOAt, 70 mg, 0.515 mmol) in N,N-dimethylformamide (3 mL) under nitrogen was added a solution of 2-(3-amino-propoxy)-5-((2S)-hydroxy-3-isopropylamino-propoxy)-benzonitrile (176 mg, 0.573 mmol) in N,N-dimethylformamide (3 mL). The reaction mixture was stirred at ambient temperature for 4 hours, diluted with water (20 mL) and washed with ethyl acetate (40 mL). The aqueous layer was left to stand at 5-10° C. for 16 hours. The precipitate which formed was filtered off and the solid was washed with water (2×10 mL) and dried under reduced pressure at 60° C. to give 2-[2-chloro-4-(6-oxo-1,4,5,6-tetrahydro-pyridazin-3-yl)-phenoxy]-N-{3-[2-cyano-4-((2S)-hydroxy-3-isopropylamino-propoxy)-phenoxy]-propyl}-acetamide hydrochloride as a colorless powder (196 mg, 66% yield, 99% pure by LC-MS and ¹H-NMR).

The compounds of Examples 12-15 can be prepared using variations of the previously described syntheses.

### Example 12

(6-{4-[3-(4-{(2S)-2-hydroxy-3-[(methylethyl)amino]propoxy}-3 bromophenoxy)propoxy]-3-chlorophenyl}-2,4,5-trihydropyridazin-3-one) is prepared as shown in Scheme X. Following cleavage of the silyl-protected phenolic group, the hydroxyl is reacted successively with (2S)-glycidyl m-nitrobenzenesulfonate and isopropylamine to deliver the compound of Example 12.

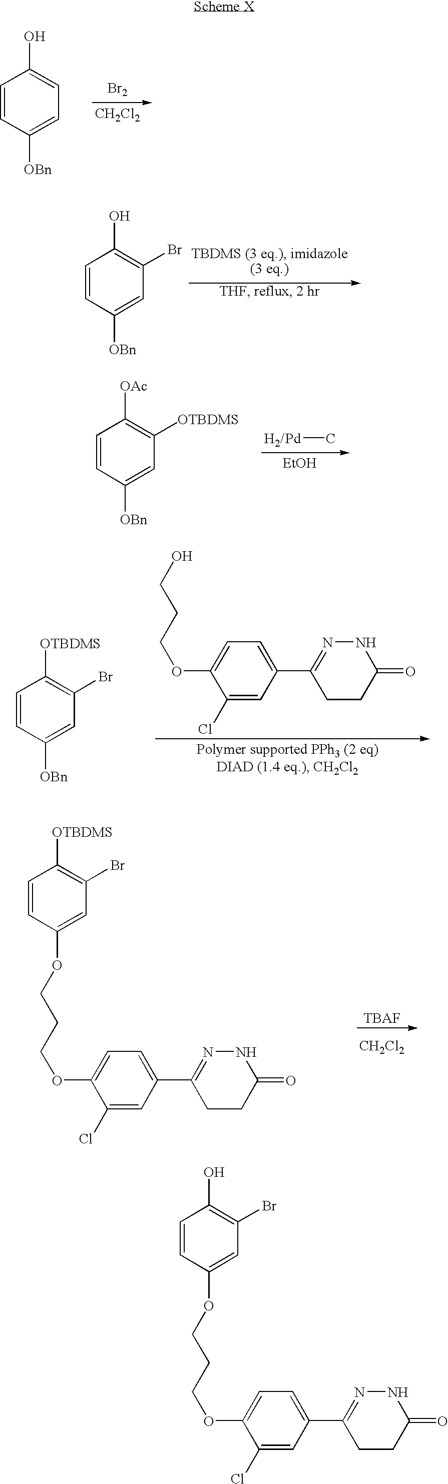

### Example 13

(2-{(2S)-2-hydroxy-3-[(methylethyl)amino]propoxy}-5-f{3-[2-chloro-4-(6-oxo(1,4,5-trihydropyridazin-3-yl))phenoxy]propoxy}benzenecarbonitrile) is prepared by reacting 3-bromo-4-(1,1,2,2-tetramethyl-1-silapropoxy)phenol, from Scheme X above, with copper cyanide in DMF to produce 5-hydroxy-2-(1,1,2,2-tetramethyl-1-silapropoxy) benzenecarbonitrile (Scheme XI). This compound is converted to Example 13 by the same sequence of steps as used for Example 12 in Scheme X.

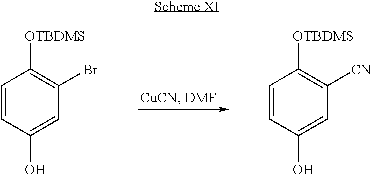

### Example 14

(6-{4-[3-(4-{(2S)-2-hydroxy-3-[(methylethyl)amino]propoxy}-2-bromophenoxy)propoxy]-3-chlorophenyl}-2,4,5-trihydropyridazin-3-one) is synthesized starting from 3-bromo-4-hydroxyphenyl acetate, as shown in Scheme XII. Following coupling of this compound with the pyridazinone glycol as described in Scheme VI for Example 8, the oxygen protecting group is removed by mild hydrolysis and the phenol is converted to Example 8 by the standard sequence of reactions already described.

### Example 15

(5-{(2S)-2-hydroxy-3-[(methylethyl)amino]propoxy}-2-{3-[2-chloro-4-(6-oxo(1,4,5-trihydropyridazin-3-yl))phenoxy]propoxy}benzenecarbonitrile) is likewise prepared by the method of Scheme XII, starting with 3-cyano-4-hydroxyphenyl acetate. 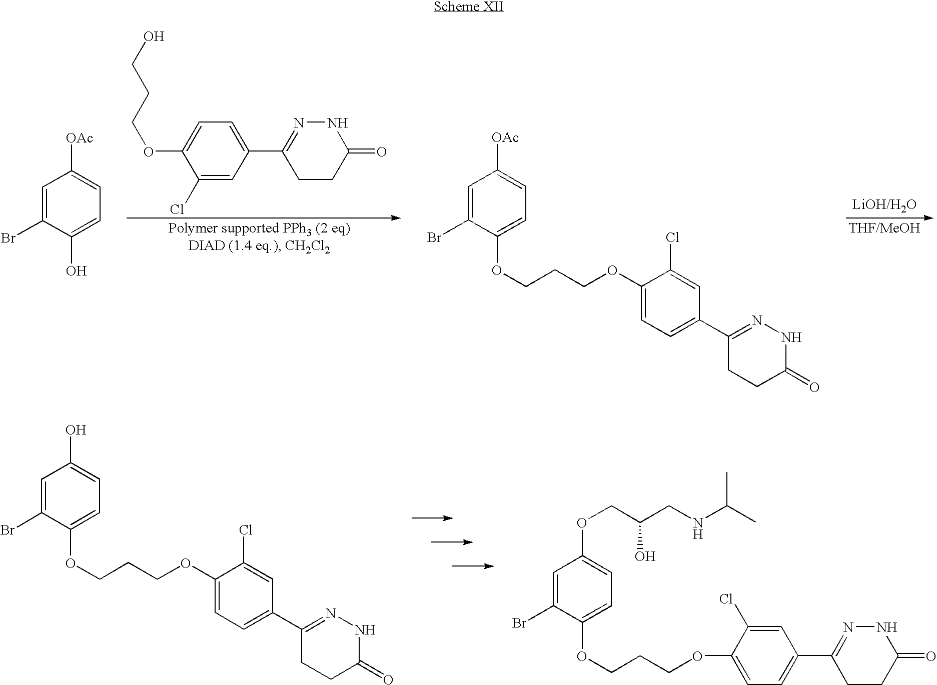
PDE-3 Inhibitory Activity

### Example 16

### Assay for Measuring cAMP PDE-3 Inhibitory Activity

Human platelet cyclic AMP phosphodiesterase is prepared according to the method of Alvarez et al., Mol. Pharmacol. 29: 554 (1986). The PDE incubation medium contains 10 mM Tris-HCl buffer, pH 7.7, 10 MM MgSO₄, and 1 μM [³H]AMP (0.2 μCi) in a total volume of 1.0 mL. Test compounds are dissolved in DMSO immediately prior to addition to the incubation medium, and the resulting mixture is allowed to stand for 10 minutes prior to the addition of enzyme. Following the addition of PDE, the contents are mixed and incubated for 10 minutes at 30° C. Three assays each are performed for each of five test compound concentrations, the mean of the determinations (n=3) at each concentration is plotted, and IC₅₀ values are determined graphically. The results are tabulated in Table I.

β-Adrenergic Receptor Binding Activity

β-Adrenergic receptor binding and blocking activity is evaluated by one or more of the methods below. The results are tabulated in Table I.

### Example 17

### Radioligand for Measuring β-Receptor Affinity

β₁-Adrenergic receptor binding is measured in human recombinant beta-l receptors expressed in CHO-REX16 cells, using [¹²⁵I] (−) Iodocyanopindolol (2000 Ci/mmol) as the radioligand, as described in Kalaria et al., J. Neurochem. 53: 1772-81 (1998), and Minneman et al., Mol. Pharmacol. 16: 34-46 (1979).

### Example 18

### Radioligand for Measuring β₂-Receptor Affinity

β₂-Adrenergic receptor binding is measured in human recombinant beta-2 receptors expressed in CHO-WT21 cells, using [¹²⁵I] (−) Iodocyanopindolol (2000 Ci/mmol) as the radioligand, as described in Kalaria et al. (1998) and Minneman et al. (1979), supra.

### Example 19

### Determination of b2-Adrenergic Blocking Activity in the Guinea Pig

Tracheal chains are prepared as described by Castillo and DeBeer, J. Pharm. Exp. Ther. 90: 104 (1947), suspended in tissue baths maintained at 37° C. containing Tyrodes solution gassed with 95% O₂-5% CO₂, and attached to an isometric force-displacement transducer. After an equilibration period of 2 hours, the preparations are induced to contract with carbachol (3×10⁻⁷ M), and relaxation is induced with cumulative dose response curves for isoproterenol first in the absence of and then in the presence of the test compound. A contact time of 10 minutes is allowed for all test compounds. Affinity constants are determined by comparing the shift in the dose-response curve for each test compound with that of isoproterenol (EC₅₀=2.3×0.2×10⁻⁸ M).

### Example 20

### Assay for Measuring Contraction-Relaxation in Guinea Pig Papillary Muscle

Male guinea pigs (400-500 g) are killed by cervical dislocation and the hearts are quickly removed, immersed in ice-cold, and oxygenated in Kreb's solution containing 113.1 mM NaCl, 4.6 mM KCl, 2.45 mM CaCl₂, 1.2 mM MgCl₂, 22.0 mM NaH₂PO₄, and 10.0 mM glucose; pH 7.4 with 95% O₂-5% CO₂. The ventricles are opened and papillary muscles are removed with chordae tandineae and a base of surrounding tissue intact. The tendinous ends of the muscles are ligated with silk thread, and the muscles are mounted in vertical, double-jacketed organ baths containing 10 mL of oxygenated Kreb's solution kept at 37° C. The tendinous end is attached to a Grass isometric force transducer, while a metal hook is inserted into the base of the muscle.

Following a 45-minute equilibration period under a 1 gram tension, control contractions are elicited by stimulating the muscle using stainless steel field electrodes at a frequency of 1.0 Hz, 2.0 ms duration. The amplitude of the stimulus is adjusted to be approximately 1.5 times the threshold amplitude sufficient to elicit a contraction of the tissues. Control contraction-relaxation cycles are recorded for 30 seconds continuously. Cumulative test drug concentrations are then injected directly into the bath while the tissue is being stimulated. Contraction-relaxation recordings are made continuously, for 30 seconds per test compound concentration. A series of washout contractions is recorded following a change of solution. Provided that the amplitude of contraction returns to that measured in control conditions, a single concentration of positive control is then tested on the tissue in the same manner as the test compound.

Contraction amplitude as well as the time courses of contraction and relaxation are quantified. All recordings are normalized against control values; statistical analysis of the results is made using t-tests or ANOVAs.

All publications, patents and patent applications identified above are herein incorporated by reference.

The invention being thus described, it will be apparent to those skilled in the art that the same may be varied in many ways without departing from the spirit and scope of the invention. Such variations are included within the scope of the invention to be claimed.

1. A compound of formula (I)
β-(Ar)ₙ-(L)ₘ-X (I)
or a pharmaceutically acceptable equivalent, an isomer or a mixture of isomers thereof, wherein:
m is chosen from 0 and 1;
n is chosen from 0 and 1;
β is chosen from a 2-amino-1-hydroxyeth-1-yl radical, N-substituted-2-amino-1-hydroxyeth-1-yl radicals, N—N-disubstituted-2-amino-1-hydroxyeth-1-yl radicals, a 3-amino-2-hydroxypropoxy radical, N-substituted-3-amino-2-hydroxypropoxy radicals, and N—N-disubstituted-3-amino-2-hydroxypropoxy radicals;
Ar is chosen from aryl radicals and heteroaryl radicals, which aryl and heteroaryl radicals are optionally substituted with one to three substituent(s) chosen from R₂, R₃, and R₄;
R₂, R₃, and R₄ are independently chosen from C₁-C₈ alkyl radicals, C₂-C₈ alkenyl radicals, C₂-C₈ alkynyl radicals, C₁-C₄ alkylthio groups, C₁-C₄ alkoxy groups, halo radicals, a nitro group, a cyano group, a trifluoromethyl group, —NR₅R₆ groups, acylaminoalkyl radicals, —NHSO₂R₁ groups and —NHCONHR₁ groups, wherein one or more —CH₂— group(s) of the alkyl, alkenyl and alkynyl radicals is/are optionally replaced with —O—, —S—, —SO₂— and/or —NR₅—, and the alkyl, alkenyl and alkynyl radicals are optionally substituted with one or more substituent(s) chosen from an oxo group and a hydroxyl group;
R₅ and R₆ are independently chosen from a lone pair of electrons, a hydrogen radical, C₁-C₈ alkyl radicals, C₂-C₈ alkenyl radicals and C₂-C₈ alkynyl radicals, wherein the alkyl, alkenyl and alkynyl radicals are optionally substituted with a substituent chosen from a phenyl radical and substituted phenyl radicals;
R₁ is chosen from C₁-C₈ alkyl radicals, C₃-C₈ cycloalkyl radicals, C₂-C₈ alkenyl radicals, C₃-C₈ cycloalkenyl radicals, C₂-C₈ alkynyl radicals and C₃-C₈ cycloalkynyl radicals;
L is chosen from a direct bond, C₁-C₁₂ alkylene radicals, C₂-C₁₂ alkenylene radicals and C₂-C₁₂ alkynylene radicals, wherein one or more —CH₂— group(s) of the alkylene, alkenylene and alkynylene radicals is/are optionally replaced with —O—, —S—, —SO₂— and/or —NR₅—, and the alkylene, alkenylene and alkynylene radicals are optionally substituted with one or more substituent(s) independently chosen from an oxo group and a hydroxyl group; and
X is chosen from moieties of formulas A-Q: 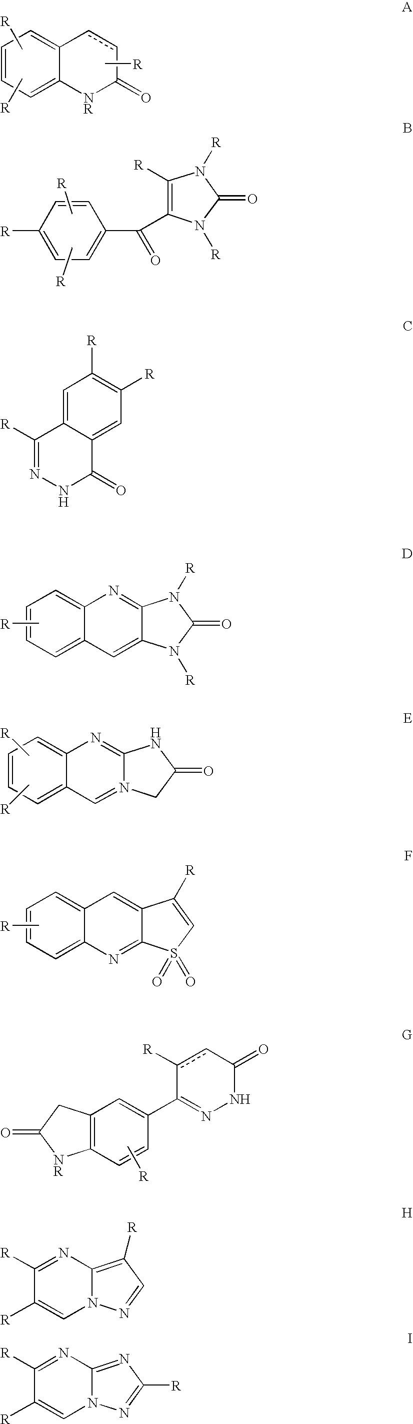 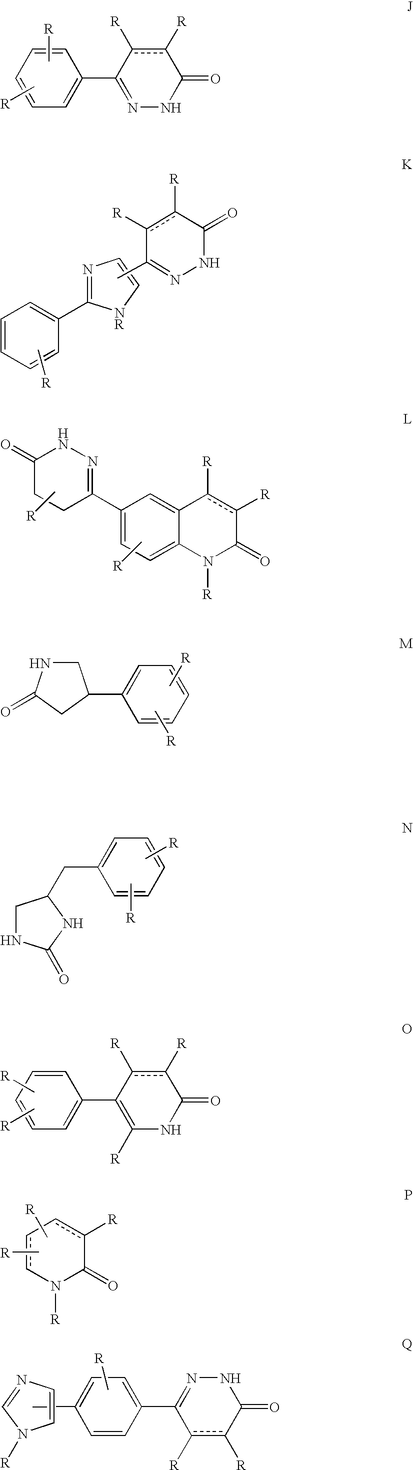
where in one R group of moieties A-Q forms a covalent bond between X and L when m is 1, or between X and Ar when n is 1 and m is 0, or between X and β when n is 0 and m is 0; and each remaining R group of moieties A-Q is independently chosen from a hydrogen radical, halo radicals, a nitro group, a cyano group, a trifluoromethyl group, an amino group, NR₅R₆ groups, C₁-C₄ alkoxy radicals, C₁-C₄ alkylthio radicals, COOR₁ radicals, C₁-C₁₂ alkyl radicals, C₂-C₁₂ alkenyl radicals and C₂-C₁₂ alkynyl radicals, wherein one or more —CH₂— group(s) of the alkyl, alkenyl and alkynyl radicals is/are optionally replaced with —O—, —S—, —SO₂— and/or —NR₅—, and the alkyl, alkenyl and alkynyl radicals are optionally substituted with one or more substituent(s) chosen from an oxo group and a hydroxyl group; and
with the following provisos:
(a) when m+n is 0, when X is chosen from A moieties, when β is chosen from a 2-amino-1-hydroxyeth-1-yl radical, N-substituted-2-amino-1-hydroxyeth-1-yl radicals, and N—N-disubstituted-2-amino-1-hydroxyeth-1-yl radicals, and
(i) when β is at position 3 or 4 of A, 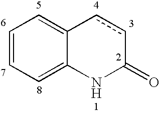
then the N-substituted-2-amino-1-hydroxyeth-1-yl radicals are not substituted with an alkyl radical, a cycloalkyl radical; an alkenyl radical; a cycloalkenyl radical, or an alkynyl radical;
and then one substituent of the N—N-disubstituted-2-amino-1-hydroxyeth-1-yl radicals is not an alkyl radical, a cycloalkyl radical; an alkenyl radical; a cycloalkenyl radical, or an alkynyl radical;
(ii) when β is at position 5 of A, then position 8 of A is not substituted with an alkoxy radical or a hydroxyl radical;
(iii) when β is at position 6 of A, position 8 of A is not substituted with an alkoxy radical, an acyloxy radical, or a hydroxyl radical; and
(iv) when β is at position 8 of A and position 5 of A is substituted with an alkoxy radical or a hydroxy radical, then the N-substituted-2-amino-1-hydroxyeth-1-yl radicals are not substituted with an alkyl radical or a cycloalkyl radical;
and then one substituent of the N—N-disubstituted-2-amino-1-hydroxyeth-1-yl radicals is not an alkyl radical or a cycloalkyl radical
(b) when m+n is 0, when X is chosen from A moieties, when β is chosen from a 3-amino-2-hydroxypropoxy radical, N-substituted-3-amino-2-hydroxypropoxy radicals, and N—N-disubstituted-3-amino-2-hydroxypropoxy radicals, and
(i) when β is at position 4 of A, then any R attached to the ring nitrogen is not a C₁-C₃ alkyl radical or a C₁-C₃ alkenyl radical;
(ii) when β is at any position 5-8 of A, then the N-substituted-3-amino-2-hydroxypropoxy radicals are not substituted with an alkyl radical; a cycloalkyl radical; an alkenyl radical; a cycloalkenyl radical; or an alkynyl radical;
and then one substituent of the N—N-disubstituted-3-amino-2-hydroxypropoxy radicals is not an alkyl radical; a cycloalkyl radical; an alkenyl radical; a cycloalkenyl radical; or an alkynyl radical;
(c) when m is 1, when n is 0, when X is chosen from A moieties, when β is chosen from a 3-amino-2-hydroxypropoxy radical, N-substituted-3-amino-2-hydroxypropoxy radicals, and N—N-disubstituted-3-amino-2-hydroxypropoxy radicals, and when β is at position 5 of A, and position 8 of A is substituted with a hydrogen radical, an alkoxy radical, or an aryloxy radical, and the R attached to the ring nitrogen is a hydrogen radical or an alkyl radical, then L is not a C₃ alkenyl radical; and
(d) when m+n is 0, when X is chosen from J moieties, when β is chosen from a 3-amino-2-hydroxypropoxy radical, N-substituted-3-amino-2-hydroxypropoxy radicals, and N—N-disubstituted-3-amino-2-hydroxypropoxy radicals, and when β is attached to the phenyl ring of J, then the N-substituted-3-amino-2-hydroxypropoxy radicals and the N—N-disubstituted-3-amino-2-hydroxypropoxy radicals are not substituted with a C₃-C₄ alkyl radical or a phenethyl radical.

2.-49. (canceled)
